(19) United States
(12) Patent Application Publication
(10) Pub. No.: US 2019/0241497 A1
(43) Pub. Date: Aug. 8, 2019

(54) METHODS FOR ANALYZING P-HYDROQUINONE LEVELS AND RATIOS

(71) Applicant: Andrew W. HINMAN, (US)

(72) Inventor: Andrew W. HINMAN, (US)

(21) Appl. No.: 16/345,677

(22) PCT Filed: Oct. 27, 2017
(86) PCT No.: PCT/US2017/058876

§ 371 Date: Apr. 26, 2019

(51) Int. Cl. C07C 69/40; C07C 69/24; C07C 69/42; C07C 69/708; G01N 33/53; G01N 33/02; C07C 69/157

(52) U.S. Cl. CPC C07C 69/40; C07C 69/24; C07C 69/42; C07C 69/157; G01N 33/5308; G01N 33/02; C07C 69/708

(57) ABSTRACT

Provided herein are compounds and methods of using the compounds for determining levels of, for example, para-hydroquinones in a sample.

### FIELD

Provided herein are methods of measuring stable para-hydroquinone derivatives in a sample.

### BACKGROUND

Para-quinones and para-hydroquinones are important biological molecules having roles in energy transfer, energy regulation, energy sensing, cell signaling, and metabolism. The concentrations of para-quinones (p-Q) and their corresponding para-hydroquinones (p-HQ) are intimately related via equilibrium processes between p-Q and p-HQ. The ratio of p-HQ to total content of p-Q and p-HQ encodes information relevant to the function of p-Q, and information relevant to levels of enzymes, cofactors, oxidative stress, and metabolic activity.

However, measurement and quantification of certain p-HQ in a sample, such as a biological sample, may be difficult by standard methods (e.g., standard mass spectrometry) due to the oxidative instability of p-HQ (i.e., auto-oxidation to the corresponding p-Q when exposed to air). For example, when the structural integrity of a cell becomes compromised, p-HQ oxidizes to p-Q. As a result, concentrations of p-HQ are typically transient and measurements of p-HQ as a component of the total content of p-HQ and p-Q levels are unreliable and not recorded. In contrast to p-HQ, p-Q species are chemically stable, and therefore, readily measured by mass spectrometry. However, because the time between sample collection and analysis varies from study to study, reported p-Q levels may contain variable contributions from p-HQ which has been transformed to p-Q by auto-oxidation.

In addition to measuring levels and ratios of p-Q and p-HQ compounds, useful information may be gained by using the Nernst equation to determine electrochemical potentials in the chemical equilibrium between p-HQ and p-Q. The Nernst equation allows for an electrochemical potential to be determined for any two compounds that equilibrate via electron transfer. The electrochemical potential for the equilibria between p-HQ and p-Q may provide valuable insights for certain biological processes.

What is needed are methods to readily assess levels and ratios for p-HQ, p-Q, and total quinone (TQ), for example, p-HQ/TQ, among other ratios, in a sample. Such methods would allow for analysis of a redox state in a sample at a static point in time, e.g., for assessment of whether a treatment is needed. Provided herein are methods of rapidly derivatizing p-HQ into a more stable form (p-dHQ) for analyzing the concentrations for p-HQ, p-Q, and TQ, and corresponding ratios with respect to each.

### SUMMARY OF THE INVENTION

Provided herein are compounds and methods of using the compounds for determining levels of, for example, para-hydroquinones in a sample.

In one aspect, provided is a compound selected from the group consisting of:

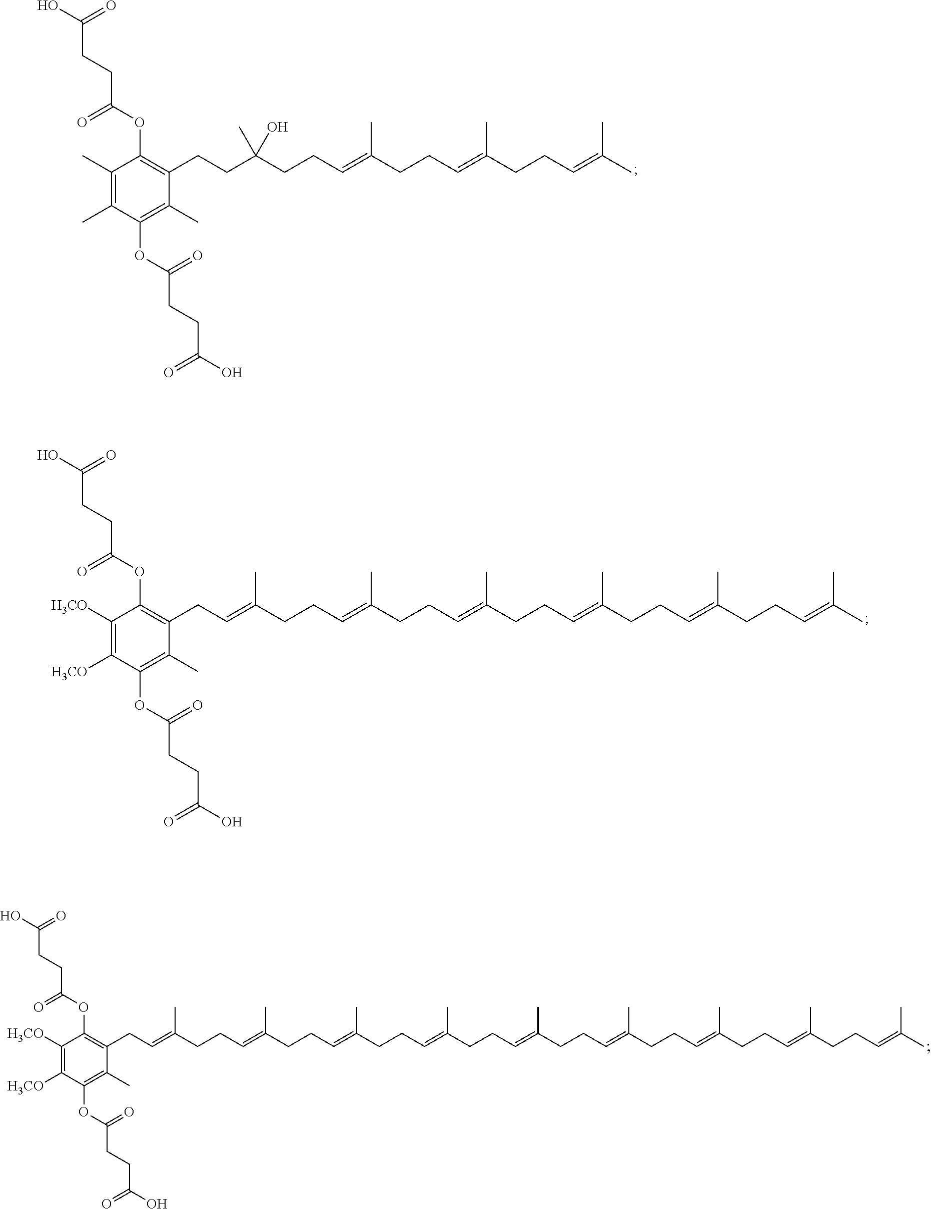 [SMILES: CC(C)=CCC/C(C)=C/CC/C(C)=C/CCC(C)(O)CCc1c(C)c(OC(=O)CCC(=O)O)c(C)c(C)c1OC(=O)CCC(=O)O ; COc1c(OC(=O)CCC(=O)O)c(C)c(C/C=C(\C)CC/C=C(\C)CC/C=C(\C)CC/C=C(\C)CC/C=C(\C)CCC=C(C)C)c(OC(=O)CCC(=O)O)c1OC ; COc1c(OC(=O)CCC(=O)O)c(C)c(C/C=C(\C)CC/C=C(\C)CC/C=C(\C)CC/C=C(\C)CC/C=C(\C)CC/C=C(\C)CC/C=C(\C)CC/C=C(\C)CCC=C(C)C)c(OC(=O)CCC(=O)O)c1OC]

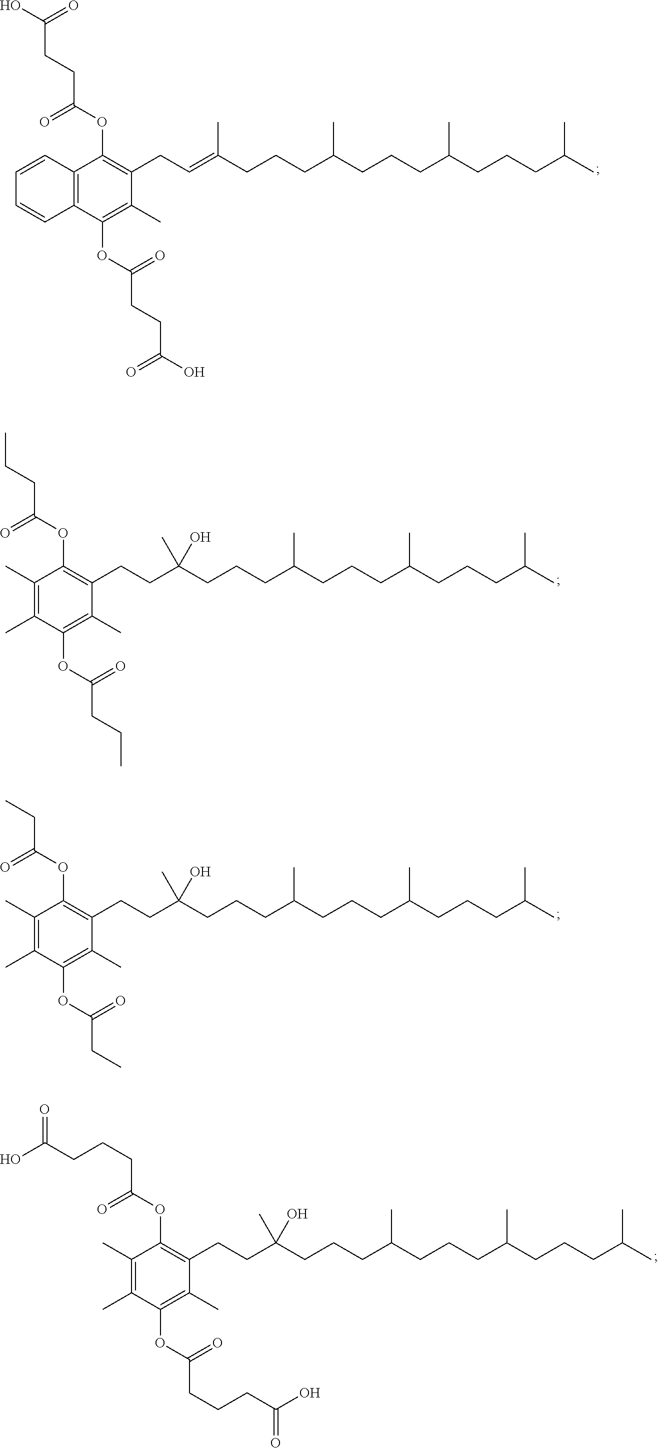

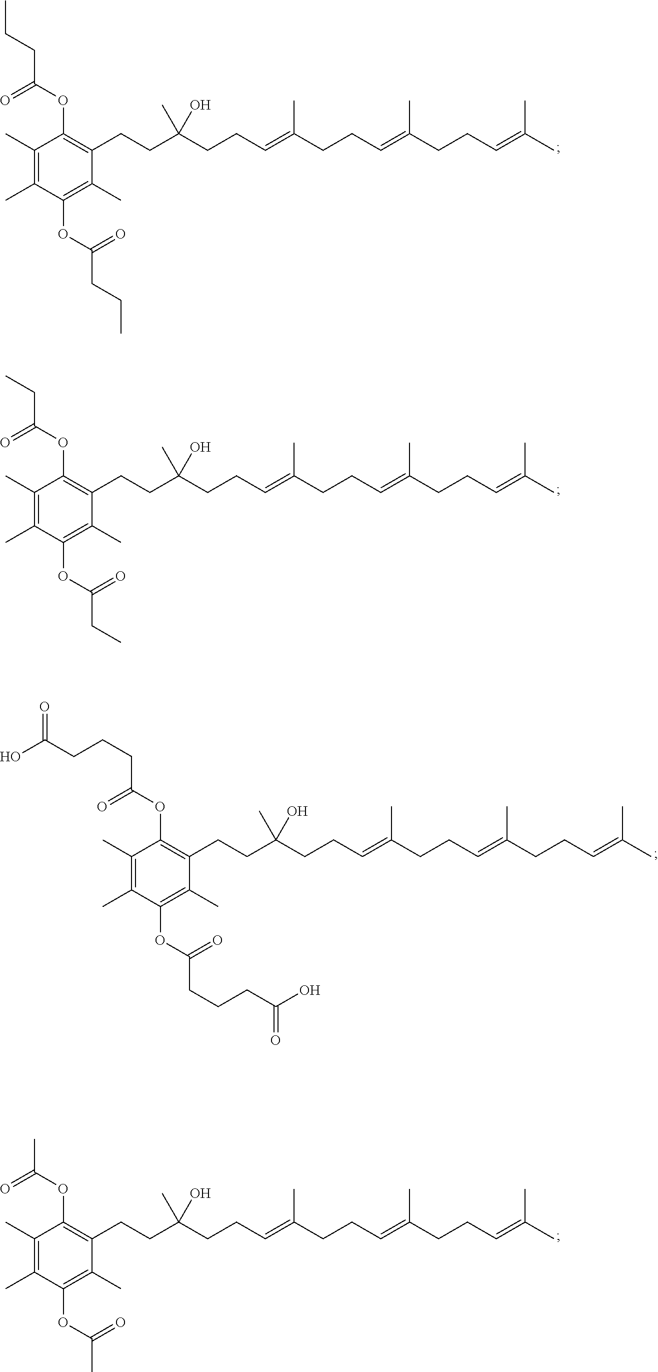

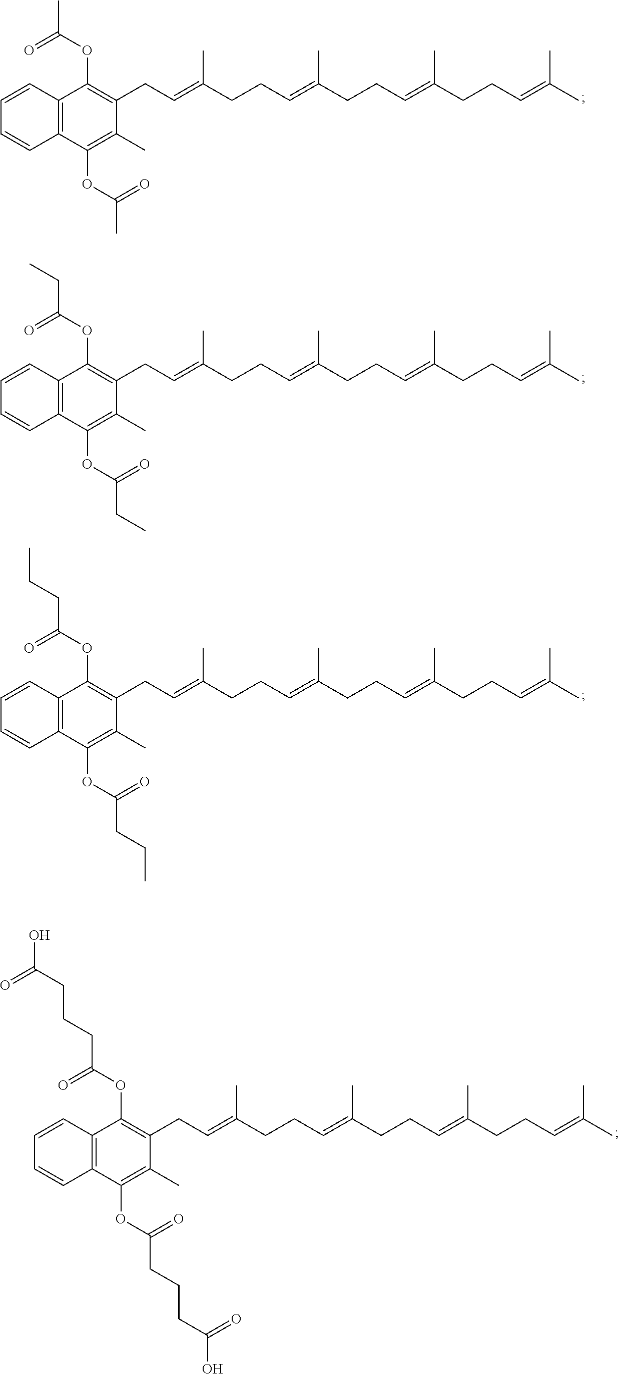 [SMILES: CC(=O)Oc1c(C)c(C/C=C(\C)CC/C=C(\C)CC/C=C(\C)CCC=C(C)C)c(OC(C)=O)c2ccccc12 ; CCC(=O)Oc1c(C)c(C/C=C(\C)CC/C=C(\C)CC/C=C(\C)CCC=C(C)C)c(OC(=O)CC)c2ccccc12 ; CCCC(=O)Oc1c(C)c(C/C=C(\C)CC/C=C(\C)CC/C=C(\C)CCC=C(C)C)c(OC(=O)CCC)c2ccccc12 ; CC(C)=CCC/C(C)=C/CC/C(C)=C/CC/C(C)=C/Cc1c(C)c(OC(=O)CCCC(=O)O)c2ccccc2c1OC(=O)CCCC(=O)O]

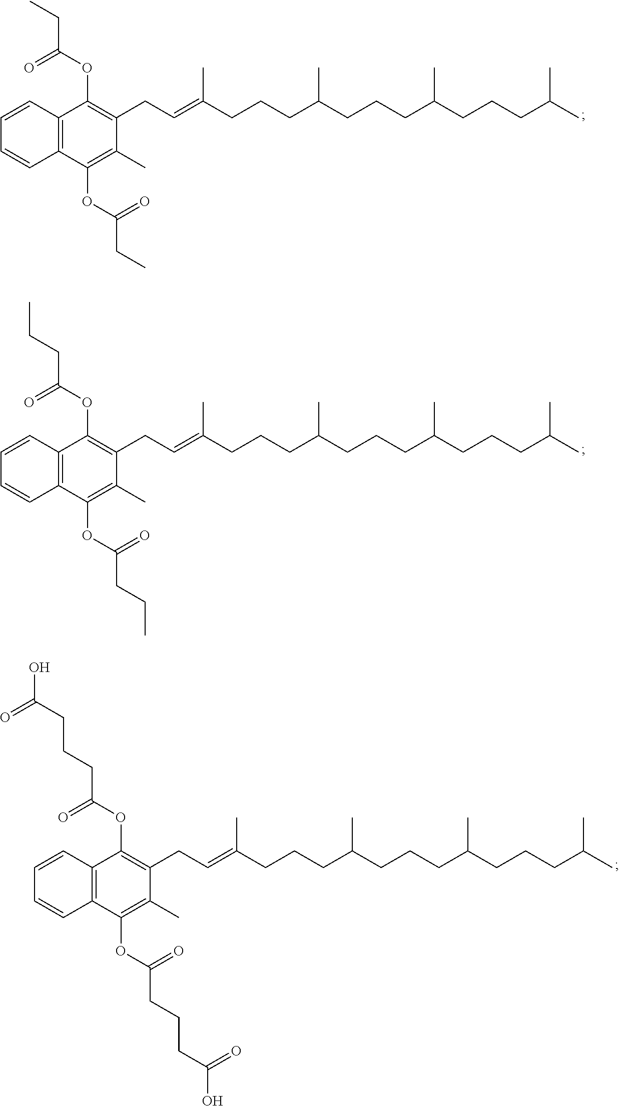 [SMILES: CCC(=O)Oc1c(C)c(C/C=C(\C)CCCC(C)CCCC(C)CCCC(C)C)c(OC(=O)CC)c2ccccc12 ; CCCC(=O)Oc1c(C)c(C/C=C(\C)CCCC(C)CCCC(C)CCCC(C)C)c(OC(=O)CCC)c2ccccc12 ; C/C(=C\Cc1c(C)c(OC(=O)CCCC(=O)O)c2ccccc2c1OC(=O)CCCC(=O)O)CCCC(C)CCCC(C)CCCC(C)C]

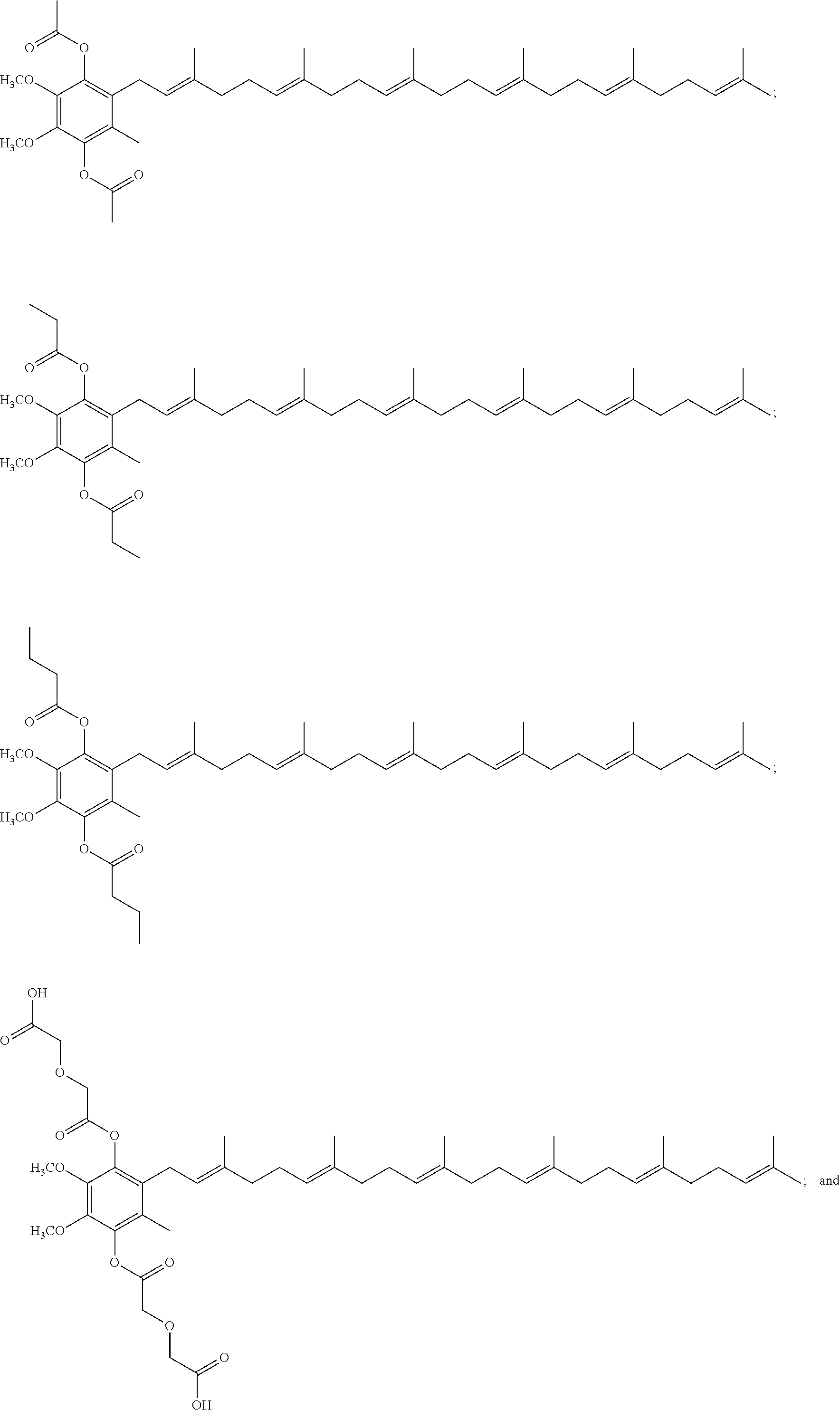 [SMILES: COc1c(OC(C)=O)c(C)c(C/C=C(\C)CC/C=C(\C)CC/C=C(\C)CC/C=C(\C)CC/C=C(\C)CCC=C(C)C)c(OC(C)=O)c1OC ; CCC(=O)Oc1c(C)c(C/C=C(\C)CC/C=C(\C)CC/C=C(\C)CC/C=C(\C)CC/C=C(\C)CCC=C(C)C)c(OC(=O)CC)c(OC)c1OC ; CCCC(=O)Oc1c(C)c(C/C=C(\C)CC/C=C(\C)CC/C=C(\C)CC/C=C(\C)CC/C=C(\C)CCC=C(C)C)c(OC(=O)CCC)c(OC)c1OC ; COc1c(OC(=O)COCC(=O)O)c(C)c(C/C=C(\C)CC/C=C(\C)CC/C=C(\C)CC/C=C(\C)CC/C=C(\C)CCC=C(C)C)c(OC(=O)COCC(=O)O)c1OC]

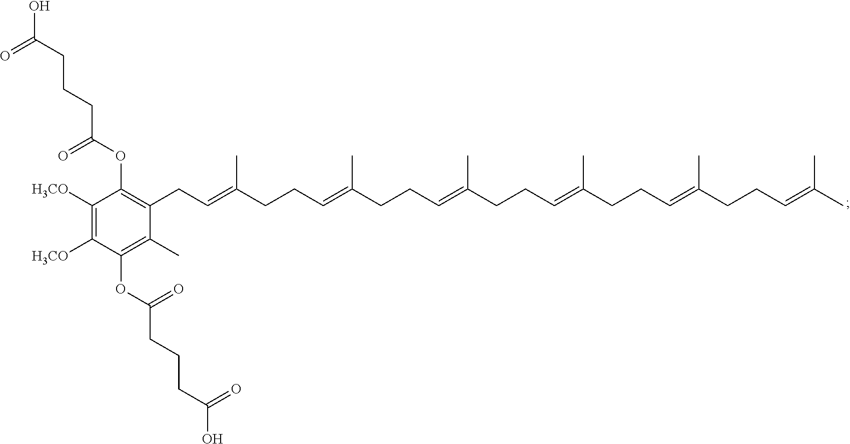 [SMILES: COc1c(OC(=O)CCCC(=O)O)c(C)c(C/C=C(\C)CC/C=C(\C)CC/C=C(\C)CC/C=C(\C)CC/C=C(\C)CCC=C(C)C)c(OC(=O)CCCC(=O)O)c1OC]

or
a stereoisomer, a mixture of stereoisomers, and/or a salt thereof.

In another aspect, provided is a method of determining the level of one or more para-hydroquinones in a first sample, the method comprising: acylating the one or more para-hydroquinones in the first sample to one or more para-hydroquinone derivatives by treatment with an alkyl carboxylic acid, an activated alkyl carboxylic acid, an alkyl anhydride, or a cyclic anhydride; and determining the level of the one or more para-hydroquinone derivatives in the first sample.

In another aspect, provided is a method of determining the ratio or reverse ratio of one or more para-hydroquinones to one or more corresponding para-quinones in a first sample, comprising:
(i) determining the level of one or more para-hydroquinone derivatives of the one or more para-hydroquinones in the first sample; and
(ii) determining one or more of the following (a)-(d):
   (a) the sum of the level of the one or more para-hydroquinone derivatives and the level of the one or more corresponding para-quinones in the first sample;
   (b) the level of one or more corresponding para-quinones in the first sample;
   (c) the level of total quinones in the first sample; and
   (d) the level of one or more corresponding para-quinones in an optional second sample taken at the same time as the first sample, wherein the one or more para-hydroquinones in the second sample were allowed to auto-oxidize to the one or more corresponding para-quinones; and
(iii) determining a ratio or reverse ratio of one or more of the following (a)-(d):
   (a) the level of the one or more para-hydroquinone derivatives in the first sample to the sum of the level of the one or more para-hydroquinone derivatives and the level of the one or more corresponding para-quinones in the first sample;
   (b) the level of the one or more para-hydroquinone derivatives in the first sample to the level of the one or more corresponding para-quinones in the first sample;
   (c) the level of the one or more para-hydroquinone derivatives in the first sample to the level of the total quinones in the first sample; and
   (d) the level of the one or more para-hydroquinone derivatives in the first sample to the level of the one or more corresponding para-quinones in the second sample.

### BRIEF DESCRIPTION OF THE DRAWING

FIG. 1 shows a plot of p-dHQ/(p-Q+p-dHQ) overlaid with OD yeast growth.

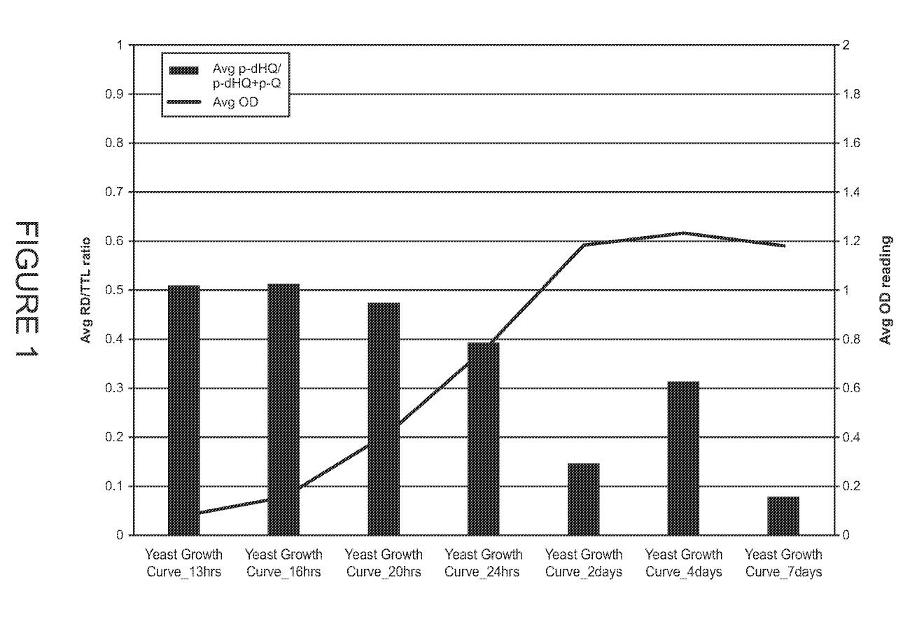

### DETAILED DESCRIPTION OF THE INVENTION

Certain para-hydroquinones are inherently unstable and oxidize to the corresponding para-quinones readily in air, rendering the direct measurement of para-hydroquinones more difficult. Because this process is dependent on the availability of atmospheric oxygen, oxidation rates will vary dramatically depending on experimental conditions such as container size or volume, solvent, handling time, and integrity of the container seal. Advantageously, the instant disclosure provides methods of measuring one or more para-hydroquinone derivative level(s) in a sample by chemically treating the one or more para-hydroquinone(s) in a sample with a reagent that traps the one or more para-hydroquinone(s), and therefore, preserves or stabilizes the level(s) of the one or more para-hydroquinone(s) for subsequent analysis. In certain embodiments, the sample may be a biological sample, thus offering the possibility of analyzing endogenous level(s) of the p-hydroquinone(s) in a biological system.

### Definitions

The abbreviations used herein have their conventional meaning within the chemical and biological arts, unless otherwise specified.

As used herein, the term “about,” when used in reference to a particular recited numerical value, refers to a value that may vary from the recited value by no more than 1%, 2%, 3%, 4%, or 5%. In some embodiments, “about” means a variance of 1%. In some embodiments, “about” means a variance of 2%. In some embodiments, “about” means a variance of 3%. In some embodiments, “about” means a variance of 4%. In some embodiments, “about” means a variance of 5%. By way of further example, as used herein, the expression “about 100” includes 99 and 101 and all values in between (e.g., 99.1, 99.2, 99.3, 99.4, etc.).

As used herein, the terms “a” or “an” refer to one or more of an element, unless the context clearly dictates otherwise.

It is to be understood that the description of compounds, compositions, and methods of analysis described herein include “comprising”, “consisting of”, and “consisting essentially of” embodiments. In some embodiments, for all compositions described herein, and all methods using a compound or composition described herein, the compositions and methods can either comprise the listed components or steps, or can “consist essentially of” the listed components or steps. When a composition is described as “consisting essentially of” the listed components, the composition contains the components listed, and may contain other components which do not substantially affect the analytical method, but do not contain any other components which substantially affect the analytical method other than those components expressly listed; or, if the composition does contain extra components other than those listed which substantially affect the analytical method, the composition does not contain a sufficient concentration or amount of the extra components to substantially affect the analytical method. When a method is described as “consisting essentially of” the listed steps, the method contains the steps listed, and may contain other steps that do not substantially affect the analytical method, but the method does not contain any other steps which substantially affect the analytical method other than those steps expressly listed.

As used herein, the term “alkyl” means a saturated linear or branched hydrocarbon. The point of attachment of the alkyl group to the remainder of the molecule can be at any chemically possible location on the alkyl group. In some embodiments, an alkyl has from 1 to 12 carbon atoms (“C₁-C₁₂ alkyl”), from 6 to 12 carbon atoms (“C₆-C₁₂ alkyl”), from 1 to 10 carbon atoms (“C₁-C₁₀ alkyl”), from 1 to 8 carbon atoms (“C₁-C₈ alkyl”), from 1 to 6 carbon atoms (“C₁-C₆ alkyl”), from 1 to 4 carbon atoms (“C₁-C₄ alkyl”), from 1 to 3 carbon atoms (“C₁-C₃ alkyl”), or from 1 to 2 carbon atoms (“C₁-C₂ alkyl”). In some embodiments, non-limiting examples of “C₁-C₆ alkyl” include methyl, ethyl, n-propyl, isopropyl, n-butyl, isobutyl, sec-butyl, t-butyl, pentyl, and hexyl.

As used herein, the term “alkylene” means a divalent alkyl group, as defined herein.

As used herein, the term “alkyl anhydride” means a R—C(O)—O—C(O)—R group where each R is the same and is an alkyl group as defined herein. In some embodiments, alkyl anhydride is acetic anhydride, propionic anhydride, or butyric anhydride.

As used herein, the term “carboxylalkyl” means an alkyl group, as defined herein, substituted with a —C(O)OH.

As used herein, the term “cyclic anhydride” means a

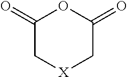

group where X is absent, —CH₂—, or −O—, i.e., succinic anhydride, glutaric anhydride, or diglycolic anhydride.

As used herein, the term “alkyl carboxylic acid” means a R—C(O)OH group where R is an alkyl, as defined herein. In some embodiments, an alkyl carboxylic acid includes, without limitation, acetic acid, propionic acid, butyric acid, and the like.

As used herein, the phrase “activated alkyl carboxylic acid” means a R—C(O)Y group where R is an alkyl group, as defined herein, and where Y is a leaving group, in some embodiments, a halo. In some embodiments, halo is bromine (Br), chlorine (Cl), fluorine (F), or iodine (I). In some embodiments, the activated alkyl carboxylic acid is prepared by treating an alkyl carboxylic acid with an activating agent such as carbonyldiimidazole (CDI), and the like.

As used herein, the terms “para-quinone,” “p-quinone,” and “p-Q” refer to compounds comprising a 1,4-benzoquinone moiety:

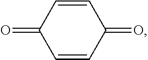

which in some embodiments includes, but is not limited to, benzoquinones, ubiquinones, napthoquinones, and anthroquinones that contain a 1,4-benzoquinone moiety.

As used herein, the terms “para-hydroquinone,” “p-hydroquinone,” and “p-HQ” refer to compounds comprising a 1,4-dihydroxyaryl moeity:

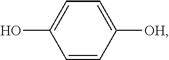

which in some embodiments include, but is not limited to, the p-hydroquinone form(s) of benzoquinones, ubiquinones, napthoquinones, and anthroquinones that contain a 1,4-benzoquinone moiety.

As used herein, the terms “para-hydroquinone derivative(s)” and “p-dHQ” refer to compounds comprising a 1,4-bis-substituted moiety:

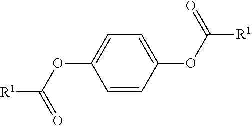

where IV is alkyl, as defined herein, carboxyalkyl, as defined herein, or -A′-O-A′-C(O)OH where A′ is alkylene, as defined herein. In some embodiments, p-HQ includes, but is not limited to, the p-hydroquinone derivatives of benzoquinones, ubiquinones, napthoquinones, and anthroquinones that contain a 1,4-benzoquinone moiety. In some embodiments, the one or more para-hydroquinone derivatives are bis-succinate derivatives (each IV is —CH₂CH₂C(O)OH), bis-glutarate derivatives (each IV is —CH₂CH₂CH₂C(O)OH), bis-diglycolate derivatives (each R¹ is —CH₂OCH₂C(O)OH)), bis-acetate derivatives (each IV is —CH₃), bis-propionate derivatives (each IV is —CH₂CH₃), or bis-butyrate derivatives (each IV is —CH₂CH₂CH₃). In some embodiments, the para-hydroquinone derivative(s) are prepared by acylating the corresponding para-hydroquinone(s) with an alkyl anhydride or a cyclic anhydride.

As used herein, the terms “total quinone” and “TQ” refer to all forms of a particular para-quinone/para-hydroquinone compound, for example, a para-hydroquinone (i.e., p-HQ) plus the corresponding derivatized para-hydroquinone (i.e., p-dHQ, as described herein) plus the corresponding para-quinone (i.e., p-Q) in a sample. For example, total quinone (TQ) refers to TQ=p-dHQ+p-HQ+p-Q.

As used herein, the term “stereoisomer” refers to geometric isomers and optically active compounds as would be appreciated by those of skill in the art. The description of compounds herein includes all stereoisomers of the compounds, including geometric isomers, diastereomers, and enantiomers, and mixtures of stereoisomers (including mixtures of geometric isomers, mixtures of enantiomers, and/or mixtures of diastereomers) in any ratio, including, but not limited to, racemic mixtures. Unless stereochemistry is explicitly indicated in a structure, the structure is intended to embrace all possible stereoisomers of the compound depicted. If stereochemistry is explicitly indicated for one portion or portions of a molecule, but not for another portion or portions of a molecule, the structure is intended to embrace all possible stereoisomers for the portion or portions where stereochemistry is not explicitly indicated.

As used herein, the term “salt” refers to ionic forms of compounds, as described herein, by treatment with acids or bases. In some embodiments, the desired salt of an acidic compound, as described herein, can be prepared by methods known to those of skill in the art by treating the compound with a base. Inorganic salts of acid compounds, as described herein, include, but are not limited to, alkali metal and alkaline earth metal salts, such as sodium salts, potassium salts, magnesium salts, and calcium salts; ammonium salts; and aluminum salts. By way of further example, organic salts of acid compounds, as described herein, include, but are not limited to, procaine, dibenzylamine, N-ethylpiperidine, N,N-dibenzylethylenediamine, and triethylamine salts. Salts of acidic compounds, as described here, with amino acids, such as lysine salts, can also be prepared.

As used herein, a “subject” refers to an animal, a mammal, avian, microorganism, cells, cell culture, or food. In some embodiments, a mammalian subject includes, without limitation, a human, chimpanzee, monkey, dog, cat, mouse, rat, cow, horse, camel, goat, pig, and sheep. In certain embodiments, the subject is a human. In certain embodiments, the subject is a bird, in some embodiments, a chicken or a duck. In certain embodiments, the subject is a microorganism. In certain embodiments, the subject is yeast. In certain embodiments, the subject is a cell culture. In certain embodiments, the subject is a food. In some embodiments, the mammalian subject has a cancer, an inflammatory disease or condition, an autoimmune disease or condition, an oxidative stress disorder, a neurodegenerative disorder, or a metabolic disorder that can be diagnosed, or monitored with methods provided herein. In some embodiments, the subject is a human that has or is suspected to have a cancer, an inflammatory disease or condition, an autoimmune disease or condition, an oxidative stress disorder, a neurodegenerative disorder, or a metabolic disorder.

As used herein, the “sample” is obtained from a subject, as defined herein. In certain embodiments, the sample is a biological sample obtained from a subject selected from the group consisting of a mammal, microorganism, cells, and cell culture. In some embodiments, the biological sample is obtained from cells, cell culture, a microorganism, a mammalian organ, mammalian tissue, or a mammalian bodily fluid. In some embodiments, cells may include, without limitation, cell cultures and cell samples. In some embodiments, cell cultures may include, without limitation, bacterial cultures and yeast cultures. By way of further example, microorganisms include, without limitation, yeast. By way of further example, mammalian bodily fluids include, without limitation, serum, plasma, cell lysates, and whole blood. In certain embodiments, the sample is a non-biological sample. In some embodiments, the sample is a food sample.

One or more para-quinone or a para-hydroquinone compounds may be administered to an animal (e.g., orally, parenterally, intravenously, etc.), and one or more portions of the animal's body may be evaluated using the methods described herein. While certain enzymes in the body convert a para-quinone to a para-hydroquinone, other species present in cells or tissues (e.g., free radicals and oxidizing species) may convert the para-hydroquinone form back to the para-quinone form. Accordingly, the equilibrium between the quinone and hydroquinone forms in a living animal, organ, tissue, or cell may reflect the redox state of the animal, organ, tissue, or cell. In some embodiments, the methods are used for evaluating in an animal, organ, tissue, or cell the oxidative stress, oxidative damage, energy level, metabolic health, drug efficacy, disease state, and the like in the animal, organ, tissue, or cell. In some embodiments, the redox state of an animal, organ, tissue, or cell having various diseases such as Parkinson's Disease, Alzheimer's Disease, and the like may be evaluated using the methods described herein.

In some embodiments, “determining the level” of a particular para-quinone, para-hydroquinone, para-hydroquinone derivative, or total quinone includes determining the total amount (e.g., mass or moles, as determined from a calibration curve by an analyst skilled in the art) of that para-quinone, para-hydroquinone, para-hydroquinone derivative, or total quinone in the sample. In some embodiments, “determining the level” of a particular para-quinone, para-hydroquinone, para-hydroquinone derivative, or total quinone includes determining the concentration of that para-quinone, para-hydroquinone, para-hydroquinone derivative, or total quinone in the sample.

If a ratio is defined as the ratio of A to B, the term “reverse ratio,” as used herein, refers to the ratio of B to A.

In some embodiments, “determining the ratio” means determining the relative amount of one or more para-hydroquinone derivatives in a first sample to the amount of one or more of the following: a) the sum of the level of the one or more para-hydroquinone derivatives and the level of the one or more corresponding para-quinones in the first sample; b) the level of the one or more corresponding para-quinones in the first sample; c) the level of the total quinones in the first sample; and d) the level of the one or more corresponding para-quinones in a second sample. In some embodiments, “determining the reverse ratio” means determining the relative amount of one or more of the following: a) the sum of the level of the one or more para-hydroquinone derivatives and the level of the one or more corresponding para-quinones in the first sample; b) the level of the one or more corresponding para-quinones in the first sample; c) the level of the total quinones in the first sample; and d) the level of the one or more corresponding para-quinones in a second sample; to the amount of a para-hydroquinone derivative.

In certain embodiments, a subject is treated with one or more para-quinone or one or more para-hydroquinone compounds, and subjected to an equilibration for a certain time period before a sample is taken. In some or any embodiments, the equilibration time period is about 2 hours. In some or any embodiments, the equilibration time period is about 3 hours. In some or any embodiments, the equilibration time period is about 4 hours. In some or any embodiments, the equilibration time period is about 5 hours. In some or any embodiments, the equilibration time period is about 6 hours. In some or any embodiments, the equilibration time period is about 7 hours. In some or any embodiments, the equilibration time period is about 3 to about 6 hours. In some or any embodiments, the equilibration time period is about 2 to about 7 hours. In some or any embodiments, the equilibration time period is about 4 to about 5 hours. In some or any embodiments, the equilibration time period is about 5 to about 6 hours. Alternatively, the one or more para-quinones or one or more para-hydroquinones are present in the subject's diet or in the cell media. In some embodiments, native quinones (e.g., CoQ10 in human; CoQ9 in rat and Saccharomyces cerevisiae; and CoQ6 in Sacchharomyces pombe) are used. In all cases, the sample is harvested and treated with an alkyl carboxylic acid, an activated alkyl carboxylic acid, an alkyl anhydride, or a cyclic anhydride according to a protocol in the Examples. In some embodiments, the sample is harvested and treated with an alkyl anhydride or a cyclic anhydride according to a protocol in the Examples. In some embodiments, an alkyl anhydride, or a cyclic anhydride is selected from the group consisting of acetic anhydride, propionic anhydride, butyric anhydride, succinic anhydride, glutaric anhydride, and diglycolic anhydride.

The levels of the various forms of the para-quinone and para-hydroquinone compounds, for example, p-Q, p-HQ, p-dHQ, and/or TQ may be determined, in some embodiments, as an absolute amount (e.g., mass or moles), or as a concentration within the sample. In some embodiments, standard LCMS integration techniques are used to determine the level(s) of the various form(s) of the one or more para-quinone and one or more para-hydroquinone compounds. In some embodiments, the level(s) are determined as an absolute amount(s). In some embodiments, the level(s) are determined as a concentration within the sample.

In some embodiments, one or more ratios are determined. In some embodiments, the ratio of the levels of the one or more corresponding para-quinone to one or more para-hydroquinone derivatives, or the reverse ratio, is determined. In some embodiments, the ratio of the levels of the one or more para-hydroquinone derivatives to total amount of the compound in all forms (i.e., p-Q, p-HQ, and p-dHQ), or the reverse ratio, is determined. In some embodiments, the ratio of the levels of the one or more para-hydroquinone derivatives to the levels of one or more para-hydroquinone derivatives plus the one or more corresponding para-quinone, or the reverse ratio, is determined. In some embodiments, p-dHQ and p-Q are measured separately, and a ratio of p-dHQ:(p-dHQ+p-Q) may be determined. In some embodiments, p-dHQ is measured, TQ is separately measured by allowing a non-derivatized sample to auto-oxidize (e.g., under an air atmosphere) where p-Q may be determined (i.e., TQ=p-Q), and a ratio of p-dHQ:TQ may be determined.

The levels of the various forms of the compound may be determined at one or more time points, in some embodiments, after the addition of the succinic anhydride to the sample. In various embodiments, the amounts are determined at 1, 2, 3, 4, 5, 10, 15, 20, 25, 30, 35, 40, or more time points.

The quinone potential of a sample can be determined using the Nernst equation:

E=E°−(RT/nF)(ln([p-Q]/[p-dHQ]²))

where E° is the redox potential of the quinone couple under standard conditions, R is the gas constant, T is the absolute temperature (in degrees Kelvin, K), n is the number of electrons transferred in the reaction, and F is Faraday's constant. [p-Q] represents the concentration of the oxidized para-quinone form, while [p-dHQ] is the concentration of reduced form as the para-hydroquinone derivative. Since quinone redox potentials in individuals are negative, between −170 mV and −90 mV, changes in the quinone redox potential are indicated by the absolute value of the change and the direction of the change. Thus, if a subject has a quinone redox potential of −120 mV, a change in redox potential of an absolute value 10 mV more negative indicates that the redox potential of the subject has changed to −130 mV. A change in quinone redox potential of an absolute value of at least about 10 mV more negative indicates that the absolute value of the change is greater than 10, and the change is more negative. If the individual's starting quinone redox potential was −120 mV, then an example of a change in redox potential of an absolute value of at least about 10 mV more negative would be a change of the quinone redox potential to −135 mV; the absolute value of the change is 15, which is at least about the absolute value of 10, and the change to the potential is in the negative direction.

### Embodiment A

The embodiments described herein include the recited compounds as well as stereoisomers, mixtures of stereoisomers, or salts thereof. In some embodiments, the compound is

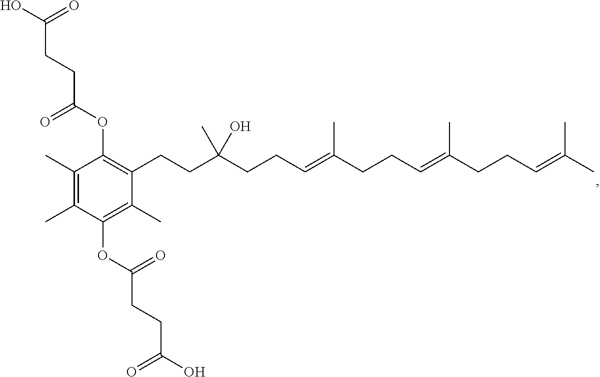 [SMILES: CC(C)=CCC/C(C)=C/CC/C(C)=C/CCC(C)(O)CCc1c(C)c(OC(=O)CCC(=O)O)c(C)c(C)c1OC(=O)CCC(=O)O]

or a stereoisomer, a mixture of stereoisomers, and/or a salt thereof. In some embodiments, the compound is

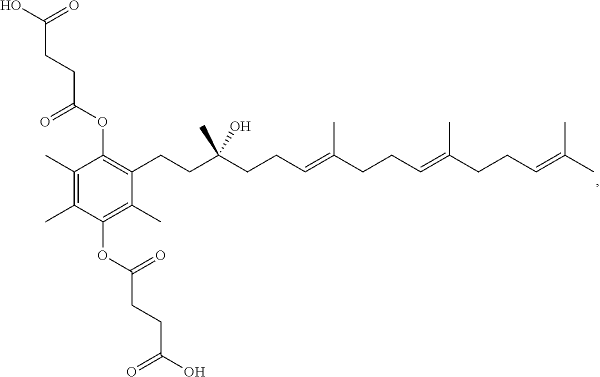 [SMILES: CC(C)=CCC/C(C)=C/CC/C(C)=C/CC[C@@](C)(O)CCc1c(C)c(OC(=O)CCC(=O)O)c(C)c(C)c1OC(=O)CCC(=O)O]

or a salt thereof. In some embodiments, the compound is

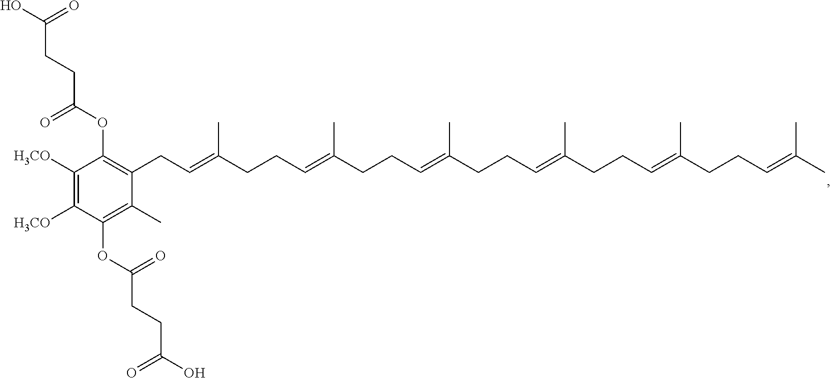 [SMILES: COc1c(OC(=O)CCC(=O)O)c(C)c(C/C=C(\C)CC/C=C(\C)CC/C=C(\C)CC/C=C(\C)CC/C=C(\C)CCC=C(C)C)c(OC(=O)CCC(=O)O)c1OC]

or a salt thereof.
In some embodiments, the compound is

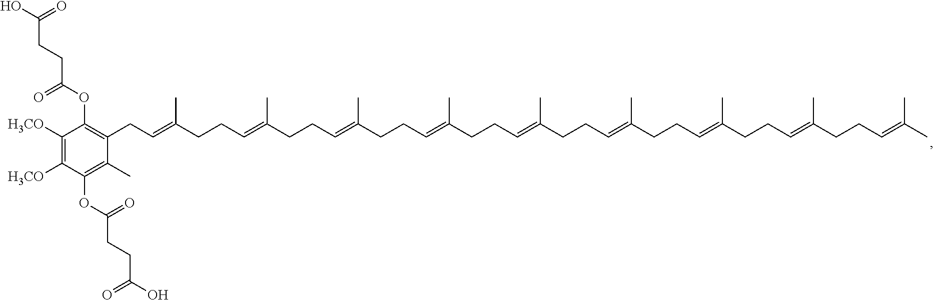 [SMILES: COc1c(OC(=O)CCC(=O)O)c(C)c(C/C=C(\C)CC/C=C(\C)CC/C=C(\C)CC/C=C(\C)CC/C=C(\C)CC/C=C(\C)CC/C=C(\C)CC/C=C(\C)CCC=C(C)C)c(OC(=O)CCC(=O)O)c1OC]

or a salt thereof. In some embodiments, the compound is

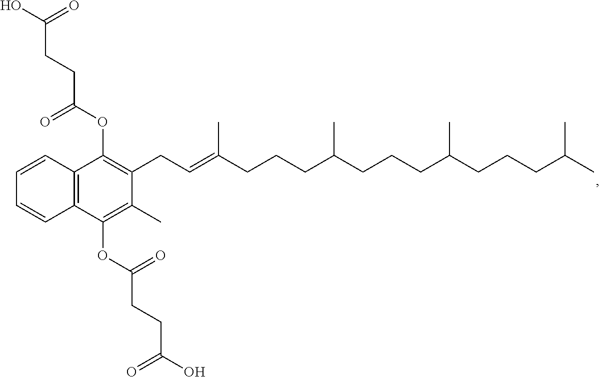 [SMILES: C/C(=C\Cc1c(C)c(OC(=O)CCC(=O)O)c2ccccc2c1OC(=O)CCC(=O)O)CCCC(C)CCCC(C)CCCC(C)C]

or a stereoisomer, a mixture of stereoisomers, and/or a salt thereof. In some embodiments, the compound is

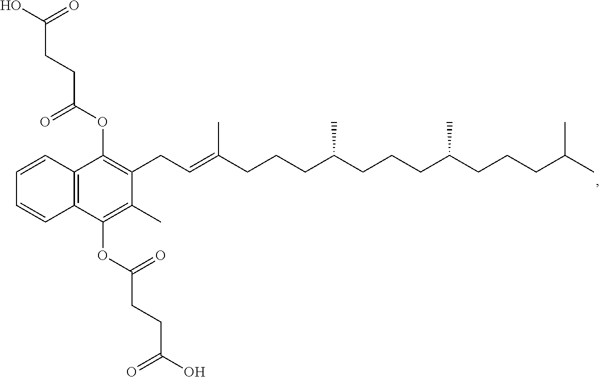 [SMILES: C/C(=C\Cc1c(C)c(OC(=O)CCC(=O)O)c2ccccc2c1OC(=O)CCC(=O)O)CCC[C@H](C)CCC[C@H](C)CCCC(C)C]

or a salt thereof. In some embodiments, the compound is

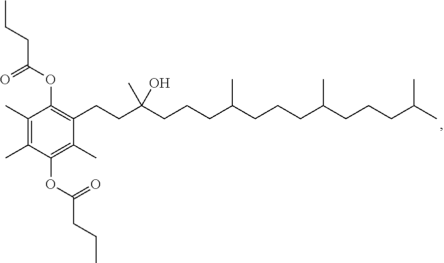 [SMILES: CCCC(=O)Oc1c(C)c(C)c(OC(=O)CCC)c(CCC(C)(O)CCCC(C)CCCC(C)CCCC(C)C)c1C]

or a stereoisomer, a mixture of stereoisomers, and/or a salt thereof. In some embodiments, the compound is

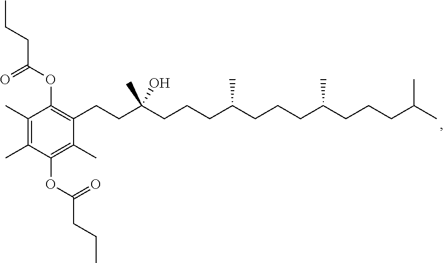 [SMILES: CCCC(=O)Oc1c(C)c(C)c(OC(=O)CCC)c(CC[C@](C)(O)CCC[C@H](C)CCC[C@H](C)CCCC(C)C)c1C]

or a salt thereof. In some embodiments, the compound is

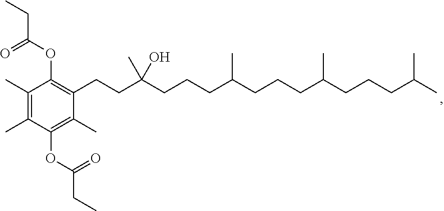 [SMILES: CCC(=O)Oc1c(C)c(C)c(OC(=O)CC)c(CCC(C)(O)CCCC(C)CCCC(C)CCCC(C)C)c1C]

or a stereoisomer, a mixture of stereoisomers, and/or a salt thereof. In some embodiments, the compound is

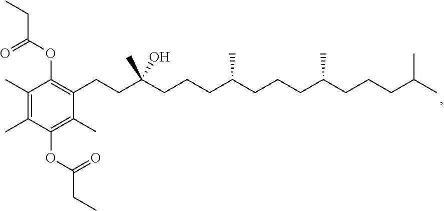 [SMILES: CCC(=O)Oc1c(C)c(C)c(OC(=O)CC)c(CC[C@](C)(O)CCC[C@H](C)CCC[C@H](C)CCCC(C)C)c1C]

or a salt thereof. In some embodiments, the compound is

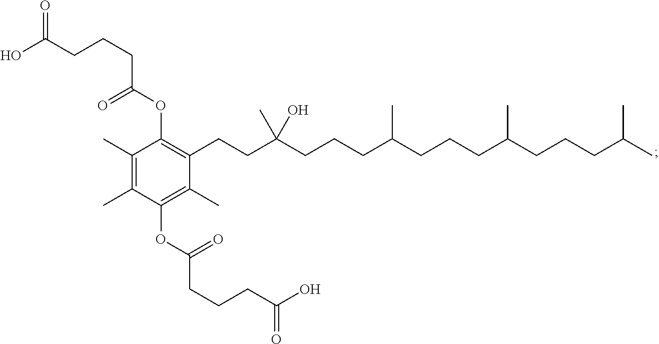 [SMILES: Cc1c(C)c(OC(=O)CCCC(=O)O)c(CCC(C)(O)CCCC(C)CCCC(C)CCCC(C)C)c(C)c1OC(=O)CCCC(=O)O]

or a stereoisomer, a mixture of stereoisomers, and/or a salt thereof. In some embodiments, the compound is

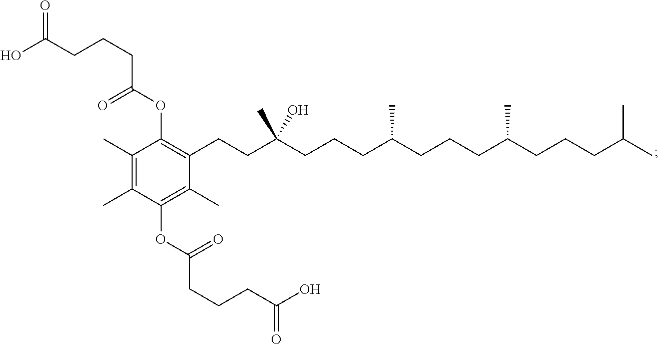 [SMILES: Cc1c(C)c(OC(=O)CCCC(=O)O)c(CC[C@](C)(O)CCC[C@H](C)CCC[C@H](C)CCCC(C)C)c(C)c1OC(=O)CCCC(=O)O]

or a salt thereof. In some embodiments, the compound is

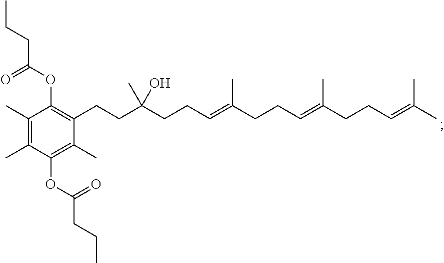 [SMILES: CCCC(=O)Oc1c(C)c(C)c(OC(=O)CCC)c(CCC(C)(O)CC/C=C(\C)CC/C=C(\C)CCC=C(C)C)c1C]

or a stereoisomer, a mixture of stereoisomers, and/or a salt thereof. In some embodiments, the compound is

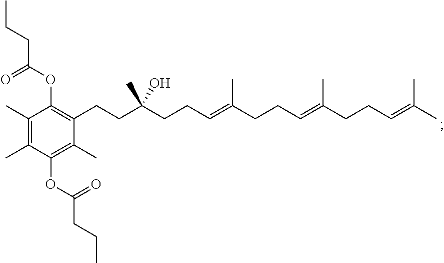 [SMILES: CCCC(=O)Oc1c(C)c(C)c(OC(=O)CCC)c(CC[C@](C)(O)CC/C=C(\C)CC/C=C(\C)CCC=C(C)C)c1C]

or a salt thereof. In some embodiments, the compound is

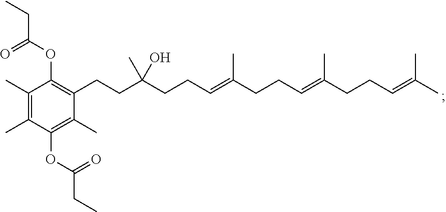 [SMILES: CCC(=O)Oc1c(C)c(C)c(OC(=O)CC)c(CCC(C)(O)CC/C=C(\C)CC/C=C(\C)CCC=C(C)C)c1C]

or a stereoisomer, a mixture of stereoisomers, and/or a salt thereof. In some embodiments, the compound is

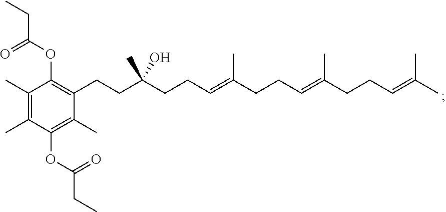 [SMILES: CCC(=O)Oc1c(C)c(C)c(OC(=O)CC)c(CC[C@](C)(O)CC/C=C(\C)CC/C=C(\C)CCC=C(C)C)c1C]

or a salt thereof. In some embodiments, the compound is

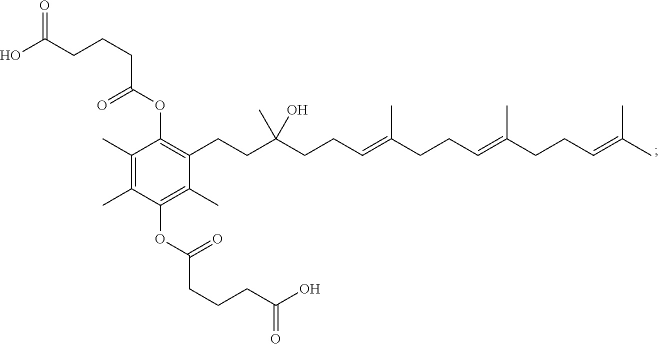 [SMILES: CC(C)=CCC/C(C)=C/CC/C(C)=C/CCC(C)(O)CCc1c(C)c(OC(=O)CCCC(=O)O)c(C)c(C)c1OC(=O)CCCC(=O)O]

or a stereoisomer, a mixture of stereoisomers, and/or a salt thereof. In some embodiments, the compound is

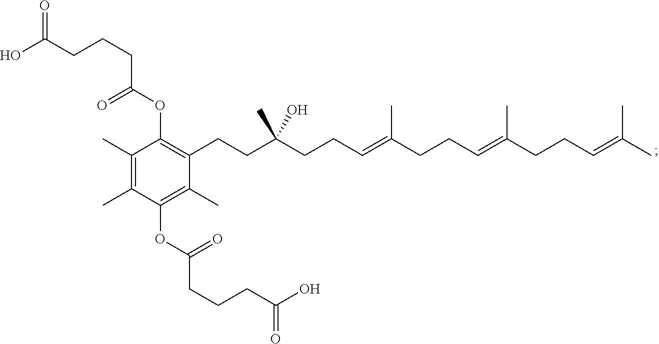 [SMILES: CC(C)=CCC/C(C)=C/CC/C(C)=C/CC[C@@](C)(O)CCc1c(C)c(OC(=O)CCCC(=O)O)c(C)c(C)c1OC(=O)CCCC(=O)O]

or a salt thereof.
In some embodiments, the compound is

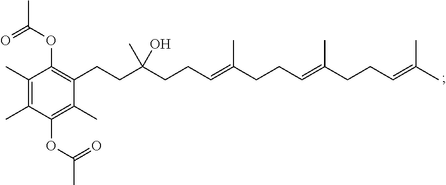 [SMILES: CC(=O)Oc1c(C)c(C)c(OC(C)=O)c(CCC(C)(O)CC/C=C(\C)CC/C=C(\C)CCC=C(C)C)c1C]

or a stereoisomer, a mixture of stereoisomers, and/or a salt thereof. In some embodiments, the compound is

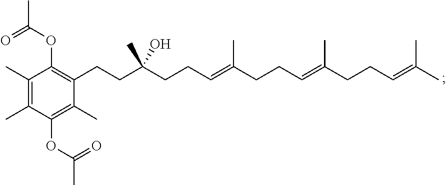 [SMILES: CC(=O)Oc1c(C)c(C)c(OC(C)=O)c(CC[C@](C)(O)CC/C=C(\C)CC/C=C(\C)CCC=C(C)C)c1C]

or a salt thereof. In some embodiments, the compound is

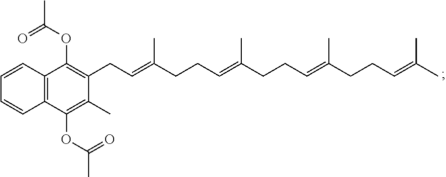 [SMILES: CC(=O)Oc1c(C)c(C/C=C(\C)CC/C=C(\C)CC/C=C(\C)CCC=C(C)C)c(OC(C)=O)c2ccccc12]

or a salt thereof. In some embodiments, the compound is

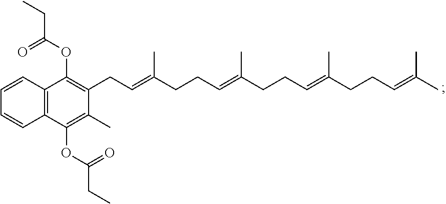 [SMILES: CCC(=O)Oc1c(C)c(C/C=C(\C)CC/C=C(\C)CC/C=C(\C)CCC=C(C)C)c(OC(=O)CC)c2ccccc12]

or a salt thereof. In some embodiments, the compound is

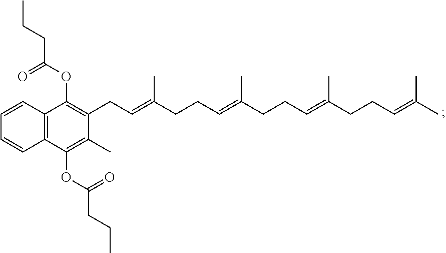 [SMILES: CCCC(=O)Oc1c(C)c(C/C=C(\C)CC/C=C(\C)CC/C=C(\C)CCC=C(C)C)c(OC(=O)CCC)c2ccccc12]

or a salt thereof. In some embodiments, the compound is

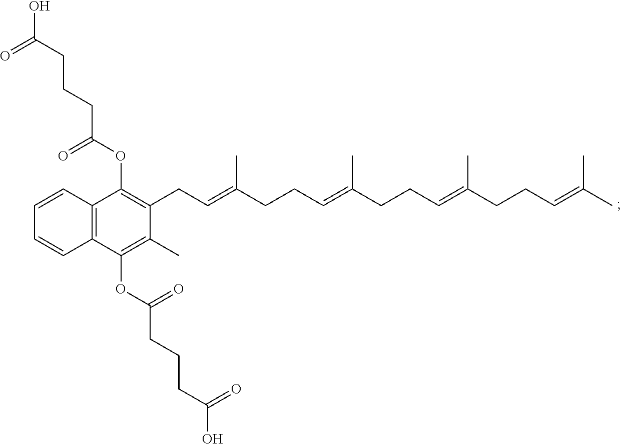 [SMILES: CC(C)=CCC/C(C)=C/CC/C(C)=C/CC/C(C)=C/Cc1c(C)c(OC(=O)CCCC(=O)O)c2ccccc2c1OC(=O)CCCC(=O)O]

or a salt thereof. In some embodiments, the compound is

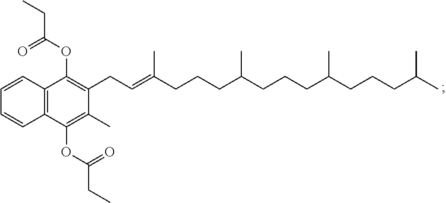 [SMILES: CCC(=O)Oc1c(C)c(C/C=C(\C)CCCC(C)CCCC(C)CCCC(C)C)c(OC(=O)CC)c2ccccc12]

or a stereoisomer, a mixture of stereoisomers, and/or a salt thereof. In some embodiments, the compound is

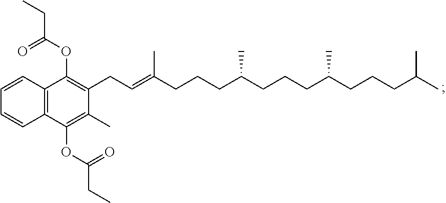 [SMILES: CCC(=O)Oc1c(C)c(C/C=C(\C)CCC[C@H](C)CCC[C@H](C)CCCC(C)C)c(OC(=O)CC)c2ccccc12]

or a salt thereof. In some embodiments, the compound is

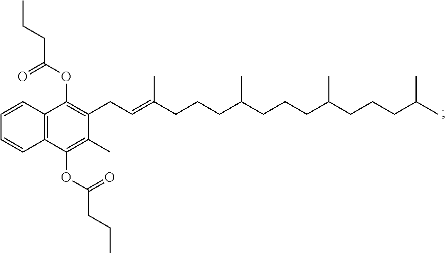 [SMILES: CCCC(=O)Oc1c(C)c(C/C=C(\C)CCCC(C)CCCC(C)CCCC(C)C)c(OC(=O)CCC)c2ccccc12]

or a stereoisomer, a mixture of stereoisomers, and/or a salt thereof. In some embodiments, the compound is

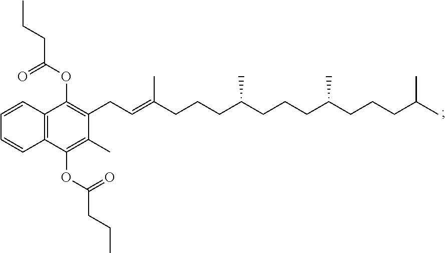 [SMILES: CCCC(=O)Oc1c(C)c(C/C=C(\C)CCC[C@H](C)CCC[C@H](C)CCCC(C)C)c(OC(=O)CCC)c2ccccc12]

or a salt thereof. In some embodiments, the compound is

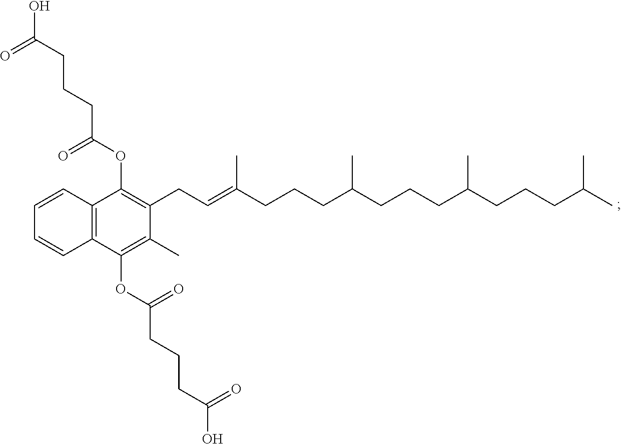 [SMILES: C/C(=C\Cc1c(C)c(OC(=O)CCCC(=O)O)c2ccccc2c1OC(=O)CCCC(=O)O)CCCC(C)CCCC(C)CCCC(C)C]

or a stereoisomer, a mixture of stereoisomers, and/or a salt thereof. In some embodiments, the compound is

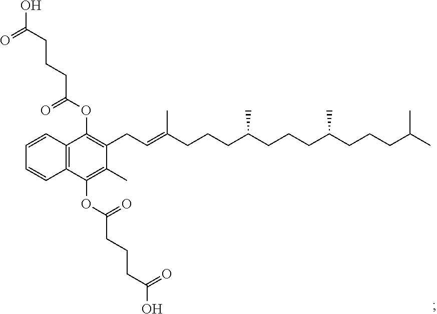 [SMILES: C/C(=C\Cc1c(C)c(OC(=O)CCCC(=O)O)c2ccccc2c1OC(=O)CCCC(=O)O)CCC[C@H](C)CCC[C@H](C)CCCC(C)C]

or a salt thereof. In some embodiments, the compound is

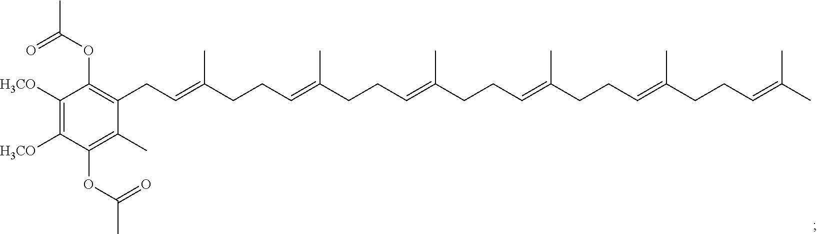 [SMILES: COc1c(OC(C)=O)c(C)c(C/C=C(\C)CC/C=C(\C)CC/C=C(\C)CC/C=C(\C)CC/C=C(\C)CCC=C(C)C)c(OC(C)=O)c1OC]

or a salt thereof.
In some embodiments, the compound is

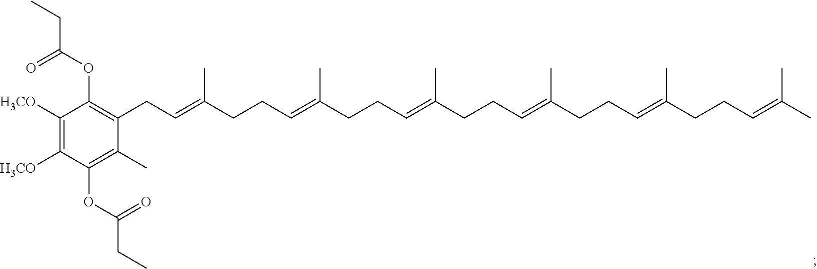 [SMILES: CCC(=O)Oc1c(C)c(C/C=C(\C)CC/C=C(\C)CC/C=C(\C)CC/C=C(\C)CC/C=C(\C)CCC=C(C)C)c(OC(=O)CC)c(OC)c1OC]

or a salt thereof.
In some embodiments, the compound is

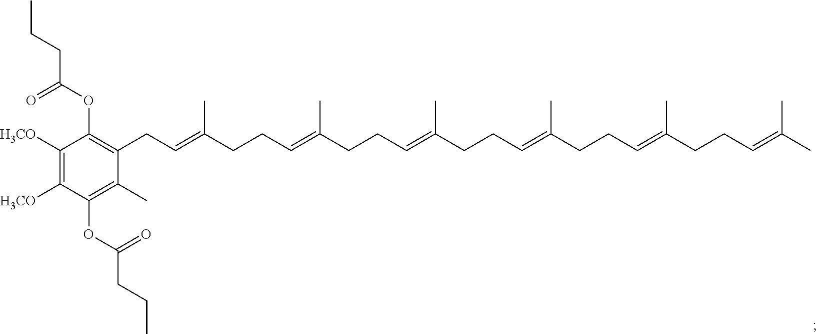 [SMILES: CCCC(=O)Oc1c(C)c(C/C=C(\C)CC/C=C(\C)CC/C=C(\C)CC/C=C(\C)CC/C=C(\C)CCC=C(C)C)c(OC(=O)CCC)c(OC)c1OC]

or a salt thereof.
In some embodiments the compound is

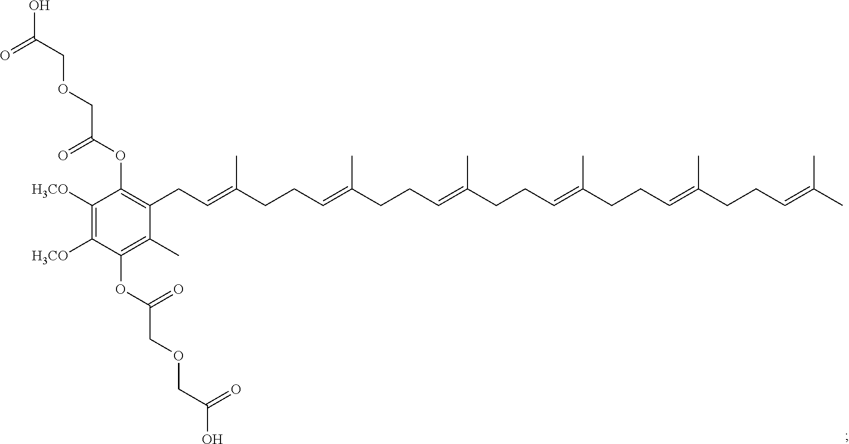 [SMILES: COc1c(OC(=O)COCC(=O)O)c(C)c(C/C=C(\C)CC/C=C(\C)CC/C=C(\C)CC/C=C(\C)CC/C=C(\C)CCC=C(C)C)c(OC(=O)COCC(=O)O)c1OC]

or a salt thereof. In some embodiments, the compound is

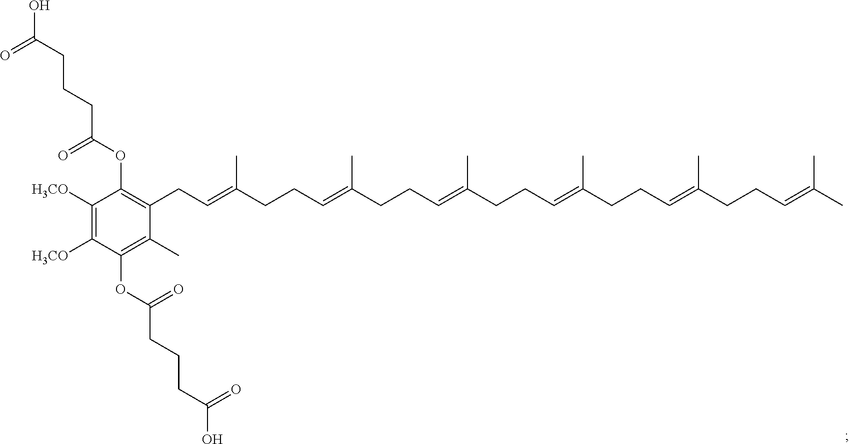 [SMILES: COc1c(OC(=O)CCCC(=O)O)c(C)c(C/C=C(\C)CC/C=C(\C)CC/C=C(\C)CC/C=C(\C)CC/C=C(\C)CCC=C(C)C)c(OC(=O)CCCC(=O)O)c1OC]

or a salt thereof

### Embodiment B

In some embodiments, provided is a compound selected from the group consisting of:

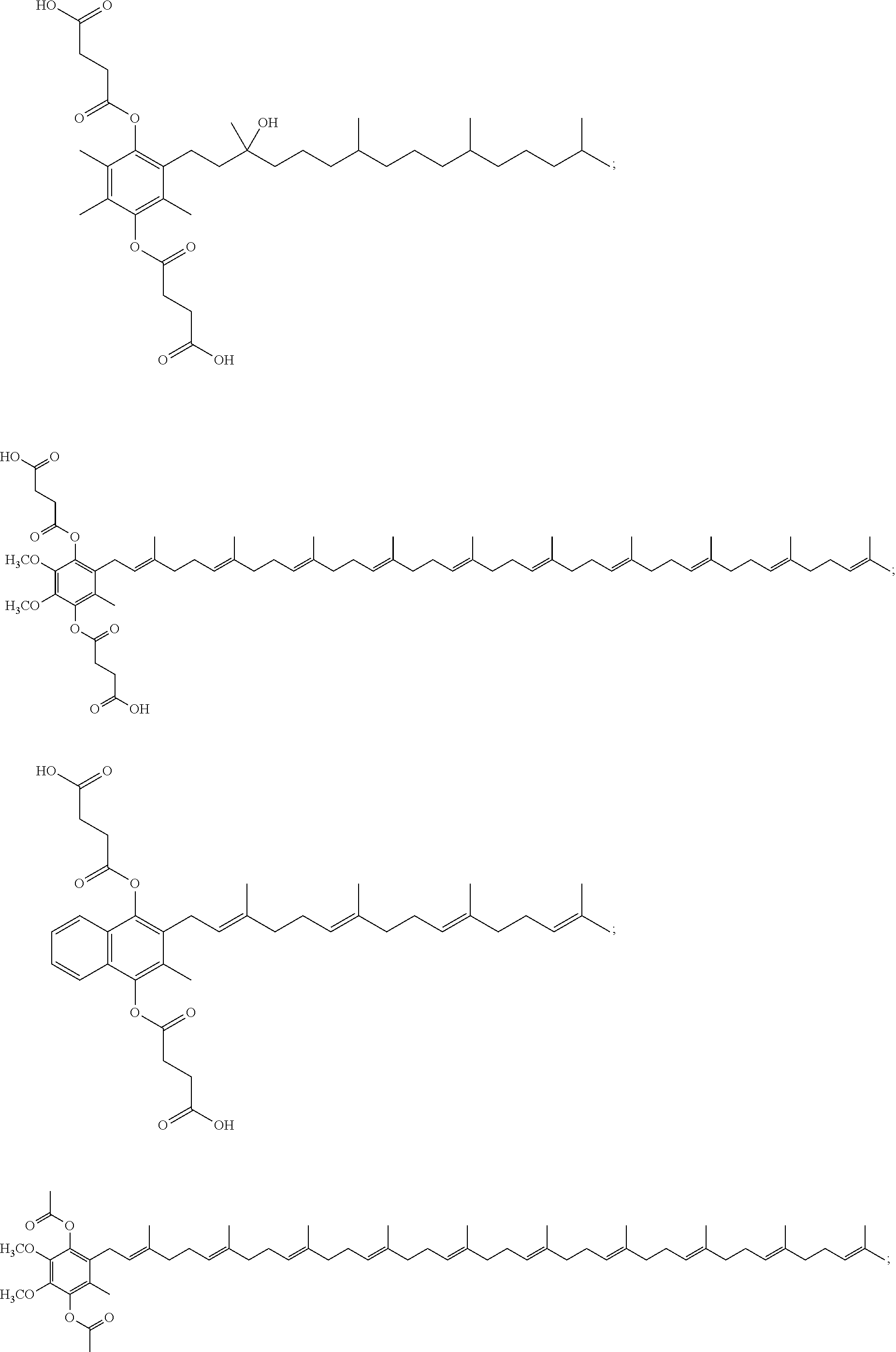 [SMILES: Cc1c(C)c(OC(=O)CCC(=O)O)c(CCC(C)(O)CCCC(C)CCCC(C)CCCC(C)C)c(C)c1OC(=O)CCC(=O)O ; COc1c(OC(=O)CCC(=O)O)c(C)c(C/C=C(\C)CC/C=C(\C)CC/C=C(\C)CC/C=C(\C)CC/C=C(\C)CC/C=C(\C)CC/C=C(\C)CC/C=C(\C)CC/C=C(\C)CCC=C(C)C)c(OC(=O)CCC(=O)O)c1OC ; CC(C)=CCC/C(C)=C/CC/C(C)=C/CC/C(C)=C/Cc1c(C)c(OC(=O)CCC(=O)O)c2ccccc2c1OC(=O)CCC(=O)O ; COc1c(OC(C)=O)c(C)c(C/C=C(\C)CC/C=C(\C)CC/C=C(\C)CC/C=C(\C)CC/C=C(\C)CC/C=C(\C)CC/C=C(\C)CC/C=C(\C)CC/C=C(\C)CCC=C(C)C)c(OC(C)=O)c1OC]

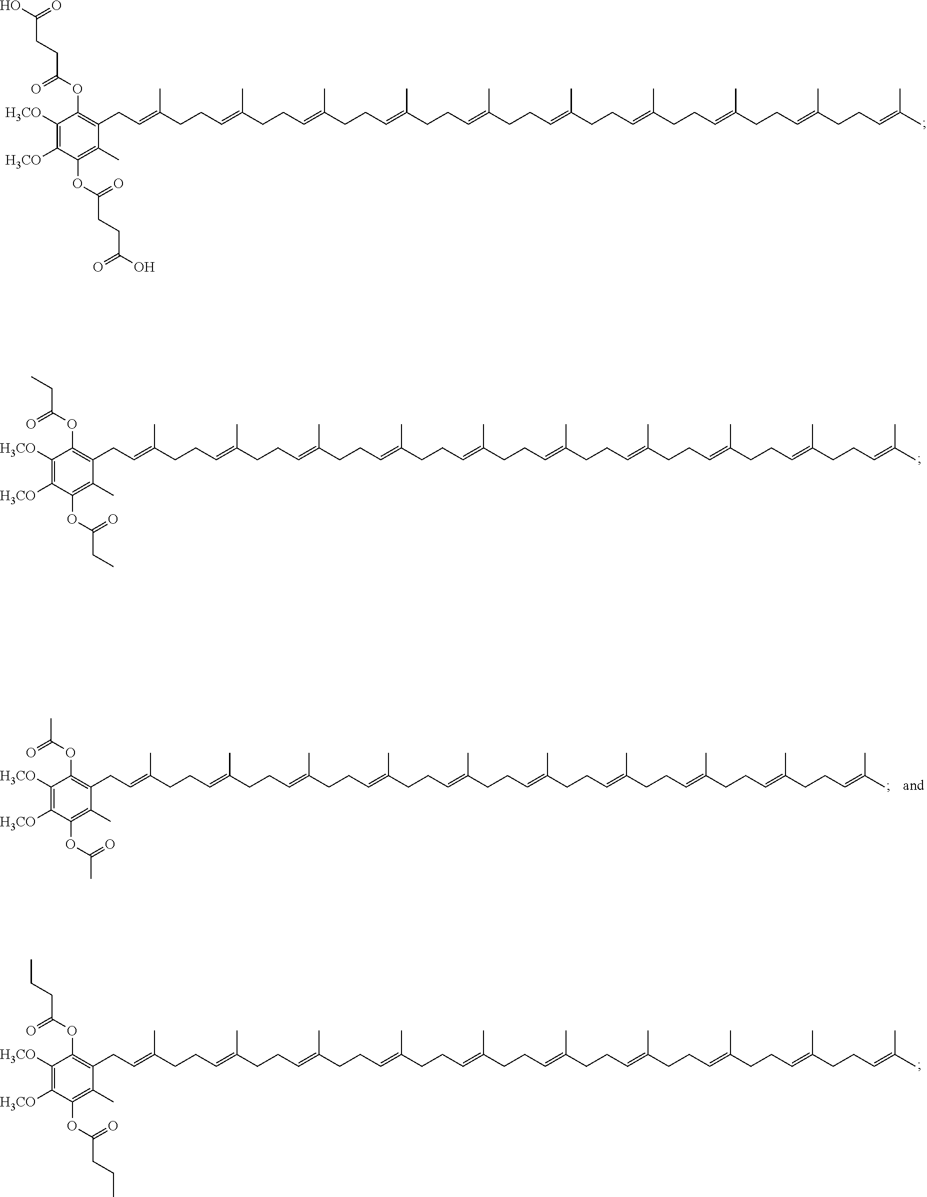

or a stereoisomer, a mixture of stereoisomers, and/or a salt thereof. In some embodiments, the compound is

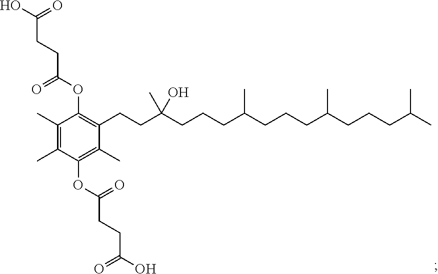 [SMILES: Cc1c(C)c(OC(=O)CCC(=O)O)c(CCC(C)(O)CCCC(C)CCCC(C)CCCC(C)C)c(C)c1OC(=O)CCC(=O)O]

or a stereoisomer, a mixture of stereoisomers, and/or a salt thereof. In some embodiments, the compound is

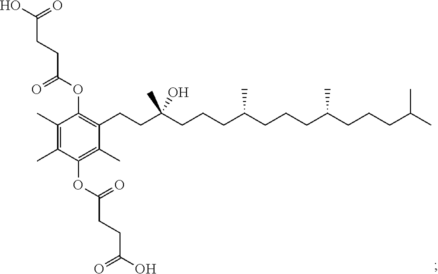 [SMILES: Cc1c(C)c(OC(=O)CCC(=O)O)c(CC[C@](C)(O)CCC[C@H](C)CCC[C@H](C)CCCC(C)C)c(C)c1OC(=O)CCC(=O)O]

or a salt thereof. In some embodiments, the compound is

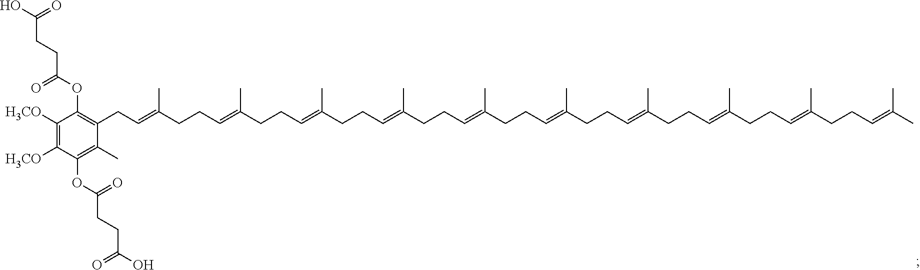 [SMILES: COc1c(OC(=O)CCC(=O)O)c(C)c(C/C=C(\C)CC/C=C(\C)CC/C=C(\C)CC/C=C(\C)CC/C=C(\C)CC/C=C(\C)CC/C=C(\C)CC/C=C(\C)CC/C=C(\C)CCC=C(C)C)c(OC(=O)CCC(=O)O)c1OC]

or a salt thereof. In some embodiments, the compound is

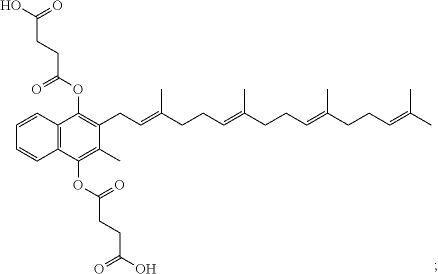 [SMILES: CC(C)=CCC/C(C)=C/CC/C(C)=C/CC/C(C)=C/Cc1c(C)c(OC(=O)CCC(=O)O)c2ccccc2c1OC(=O)CCC(=O)O]

or a salt thereof. In some embodiments, the compound is

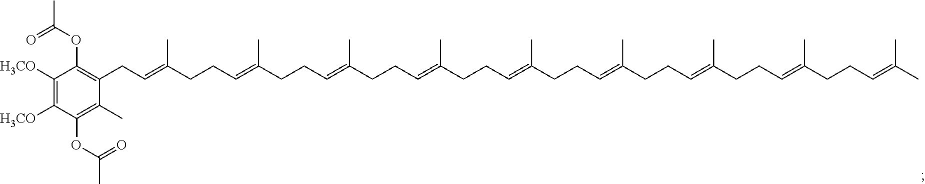 [SMILES: COc1c(OC(C)=O)c(C)c(C/C=C(\C)CC/C=C(\C)CC/C=C(\C)CC/C=C(\C)CC/C=C(\C)CC/C=C(\C)CC/C=C(\C)CC/C=C(\C)CCC=C(C)C)c(OC(C)=O)c1OC]

or a salt thereof. In some embodiments, the compound is

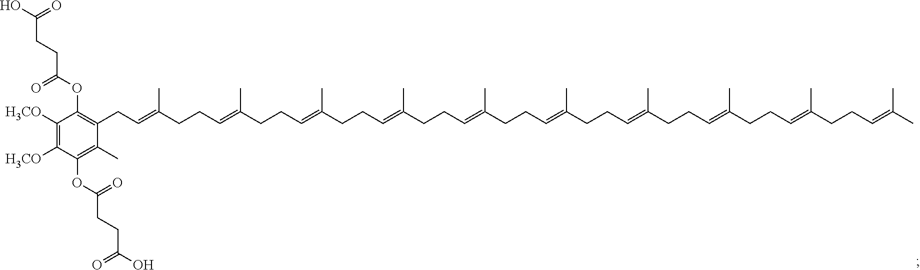 [SMILES: COc1c(OC(=O)CCC(=O)O)c(C)c(C/C=C(\C)CC/C=C(\C)CC/C=C(\C)CC/C=C(\C)CC/C=C(\C)CC/C=C(\C)CC/C=C(\C)CC/C=C(\C)CC/C=C(\C)CCC=C(C)C)c(OC(=O)CCC(=O)O)c1OC]

or a salt thereof. In some embodiments, the compound is

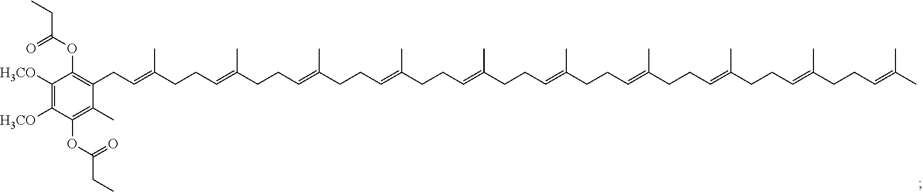 [SMILES: CCC(=O)Oc1c(C)c(C/C=C(\C)CC/C=C(\C)CC/C=C(\C)CC/C=C(\C)CC/C=C(\C)CC/C=C(\C)CC/C=C(\C)CC/C=C(\C)CC/C=C(\C)CCC=C(C)C)c(OC(=O)CC)c(OC)c1OC]

or a salt thereof. In some embodiments, the compound is

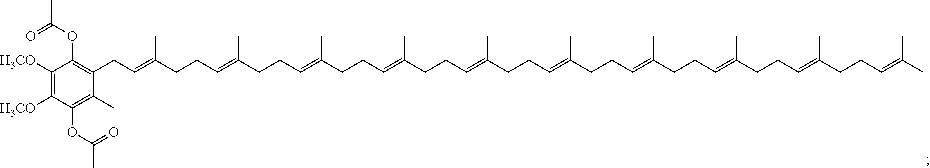 [SMILES: COc1c(OC(C)=O)c(C)c(C/C=C(\C)CC/C=C(\C)CC/C=C(\C)CC/C=C(\C)CC/C=C(\C)CC/C=C(\C)CC/C=C(\C)CC/C=C(\C)CC/C=C(\C)CCC=C(C)C)c(OC(C)=O)c1OC]

or a salt thereof. In some embodiments, the compound is

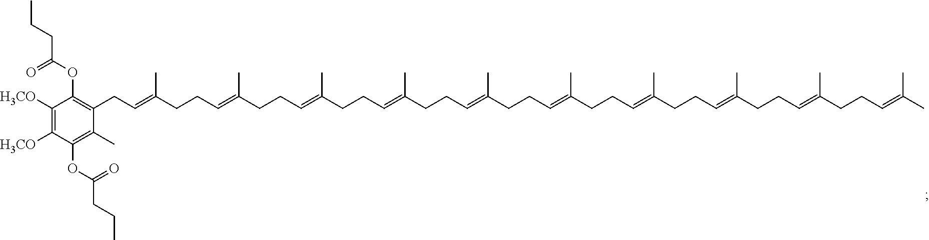 [SMILES: CCCC(=O)Oc1c(C)c(C/C=C(\C)CC/C=C(\C)CC/C=C(\C)CC/C=C(\C)CC/C=C(\C)CC/C=C(\C)CC/C=C(\C)CC/C=C(\C)CC/C=C(\C)CCC=C(C)C)c(OC(=O)CCC)c(OC)c1OC]

or a salt thereof.

In some embodiments, including any of the foregoing Embodiments A and B, the compound is not a salt. In some embodiments, including any of the foregoing Embodiments A and B, the compound is a salt. In some embodiments, including any of the foregoing Embodiments A and B, the compound is a pharmaceutically acceptable salt.

The embodiments described herein further include methods of determining the level of one or more para-hydroquinones in a first sample comprising acylating the one or more para-hydroquinones in the first sample to one or more para-hydroquinone derivatives by treatment with an alkyl carboxylic acid, an activated alkyl carboxylic acid, an alkyl anhydride, or a cyclic anhydride; and determining the level of the one or more para-hydroquinone derivatives in the first sample. In some embodiments, provided are methods of determining the level of one or more para-hydroquinones in a first sample comprising acylating the one or more para-hydroquinones in the first sample to one or more para-hydroquinone derivatives by treatment with an alkyl anhydride or a cyclic anhydride; and determining the level of the one or more para-hydroquinone derivatives in the first sample. In some embodiments, the one or more para-hydroquinone derivatives are selected from the group consisting of a bis-acetate derivative, a bis-propionate derivative, a bis-butyrate derivative, a bis-succinate derivative, a bis-glutarate derivative, and a bis-diglycolate derivative. In some embodiments, the one or more para-hydroquinone derivatives are bis-acetate derivatives. In some embodiments, the one or more para-hydroquinone derivatives arebis-propionate derivatives. In some embodiments, the one or more para-hydroquinone derivatives are bis-butyrate derivatives. In some embodiments, the one or more para-hydroquinone derivatives are bis-succinate derivatives. In some embodiments, the one or more para-hydroquinone derivatives are bis-glutarate derivatives. In some embodiments, the one or more para-hydroquinone derivatives are bis-diglycolate derivatives. In some embodiments, the method further comprises determining the level of one or more corresponding para-quinones in an optional second sample, taken at the same time as the first sample, wherein the one or more para-hydroquinones in the second sample was allowed to auto-oxidize to the one or more corresponding para-quinones. In some or any embodiments, the one or more para-hydroquinones in the second sample are the same as the one or more para-hydroquinones in the first sample. In some embodiments, auto-oxidation is performed by exposing the second sample to atmospheric air. In some or any embodiments, the one or more para-hydroquinone derivative comprises a compound selected from Embodiment A and Embodiment B. In some or any embodiments, the one or more para-hydroquinone derivative comprises a compound selected from Embodiment A. In some or any embodiments, the one or more para-hydroquinone derivative comprises a compound selected from Embodiment B. In some or any embodiments, the levels are determined by mass spectroscopy. In some or any embodiments, the levels are determined by liquid chromatography-mass spectroscopy (LC-MS). In some or any embodiments, the first and optional second samples are biological samples. In some or any embodiments, the biological samples are selected from the group consisting of a cell sample, bacterial culture, yeast culture, plasma, serum, whole blood, and a biological tissue sample. In some or any embodiments, the first sample and optional second sample are not biological samples.

In another embodiment, provided is a method of determining the ratio or reverse ratio of one or more para-hydroquinones to one or more corresponding para-quinones in a first sample, comprising:
(i) determining the level of one or more para-hydroquinone derivatives of the one or more para-hydroquinones in the first sample; and
(ii) determining one or more of the following (a)-(d):
   (a) the sum of the level of the one or more para-hydroquinone derivatives and the level of the one or more corresponding para-quinones in the first sample;
   (b) the level of one or more corresponding para-quinones in the first sample; and
   (c) the level of total quinones in the first sample; and
   (d) the level of one or more corresponding para-quinones in an optional second sample taken at the same time as the first sample, wherein the one or more para-hydroquinone in the second sample were allowed to auto-oxidize to the one or more corresponding para-quinones; and
(iii) determining a ratio or reverse ratio of one or more of the following (a)-(d):
   (a) the level of the one or more para-hydroquinone derivatives in the first sample to the sum of the level of the one or more para-hydroquinone derivatives and the level of the one or more corresponding para-quinones in the first sample;
   (b) the level of the one or more para-hydroquinone derivatives in the first sample to the level of the one or more corresponding para-quinones in the first sample;
   (c) the level of the one or more para-hydroquinone derivatives in the first sample to the level of the total quinones in the first sample; and
   (d) the level of the one or more para-hydroquinone derivatives in the first sample to the level of the one or more corresponding para-quinones in the second sample.
      In some or any embodiments, the ratio or reverse ratio of the level of the one or more para-hydroquinone derivatives in the first sample to the sum of the level of the one or more para-hydroquinone derivatives and the level of the one or more corresponding para-quinones in the first sample is determined. In some or any embodiments, the ratio or reverse ratio of the level of the one or more para-hydroquinone derivatives in the first sample to the level of the one or more corresponding para-quinones in the first sample is determined. In some or any embodiments, the ratio or reverse ratio of the level of the one or more para-hydroquinone derivatives in the first sample to the level of the total quinones in the first sample is determined. In some or any embodiments, the ratio or reverse ratio of the level of the one or more para-hydroquinone derivatives in the first sample to the level of the one or more corresponding para-quinones in the second sample is determined. In some or any embodiments, the one or more para-hydroquinone derivative comprises a compound selected from Embodiment A and Embodiment B. In some or any embodiments, the one or more para-hydroquinone derivatives comprise compounds selected from Embodiment A. In some or any embodiments, the one or more para-hydroquinone derivatives comprise compounds selected from Embodiment B. In some or any embodiments, the levels are determined by mass spectroscopy. In some or any embodiments, the levels are determined by liquid chromatography-mass spectroscopy (LC-MS). In some or any embodiments, the first and optional second samples are biological samples. In some embodiments, the biological samples are selected from the group consisting of a cell sample, bacterial culture, yeast culture, plasma, serum, whole blood, and a biological tissue sample. In some embodiments, the biological sample is a cell sample. In some embodiments, the biological sample is a bacterial culture. In some embodiments, the biological sample is a yeast culture. In some embodiments, the biological sample is plasma. In some embodiments, the biological sample is serum. In some embodiments, the biological sample is whole blood. In some embodiments, the biological sample is a biological tissue sample. In some embodiments, the first sample and optional second sample are not biological samples.

Kits

Also provided are articles of manufacture and kits containing materials useful for determining the level of one or more para-hydroquinones in a sample. Also provided are kits comprising an acylator (in some embodiments an alkyl carboxylic acid, an activated alkyl carboxylic acid, an alkyl anhydride, or a cyclic anhydride) as described herein in a suitable container, and instructions for use in determining the level of one or more para-hydroquinones in a sample. Examples of suitable containers include, but are not limited to, glass and plastic (in some embodiments, polyethylene, polypropylene, and/or polycarbonate) containers (in some embodiments, bottles or vials). Instructions may be provided in printed form or in the form of an electronic medium such as a floppy disc, CD, or DVD, or in the form of a website address where such instructions may be obtained.

### EXAMPLES

### Example 1—Alpha-Tocotrienol Hydroquinone Bis-Succinate Ester, Sample Preparation

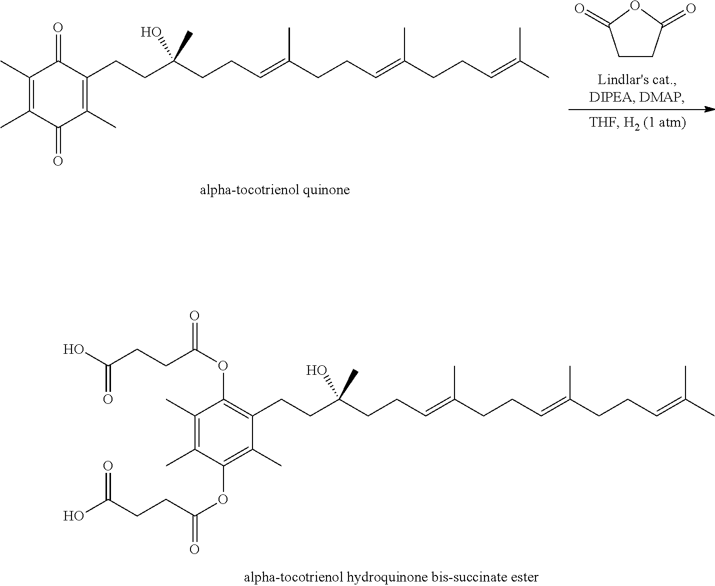 [SMILES: CC(C)=CCC/C(C)=C/CC/C(C)=C/CC[C@@](C)(O)CCC1=C(C)C(=O)C(C)=C(C)C1=O ; O=C1CCC(=O)O1 ; CC(C)=CCC/C(C)=C/CC/C(C)=C/CC[C@@](C)(O)CCc1c(C)c(OC(=O)CCC(=O)O)c(C)c(C)c1OC(=O)CCC(=O)O]

In a 20 mL scintillation vial equipped with a stir bar was added alpha-tocotrienol quinone (100 mg, 0.23 mmol), succinic anhydride (227 mg, 2.27 mmol), Lindlar's catalyst (10% wt, 5 mg), diisoproylethylamine (395 uL, 2.27 mmol), 4-dimethylaminopyridine (DMAP) (7 mg, 0.06 mmol) and tetrahydrofuran (THF) (1.5 mL, 0.15 M). After the addition was complete, H₂ gas was bubbled through the solution for 1 min and then the vessel was sealed and stirred under H₂ at room temperature. After stirring for 18 hr, the reaction mixture was filtered through a syringe filter and concentrated via rotary exaporation under vacuum at 40° C. and 5 torr. The residue was then re-dissolved with isopropyl acetate (10 mL), washed with deionized water and saturated aqueous sodium chloride (5 mL each), dried with sodium sulfate, filtered, and concentrated in vacuo. Crude product was obtained as 130 mg of a pale yellow/orange solid. Isolation was performed via a silica column (12 g column), eluting with 100% ethyl acetate (monitored at 255 nm). 40 mg of white solid was obtained (27% yield). Sample yield and purity was obtained by LC/MS and NMR. LC/MS analysis showed peak, t=4.669. (+)m/z=660.3. ¹H-NMR (400 MHz, CD₃OD, 25° C.): δ=5.10 (t, 3H), 2.94 (m, 4H), 2.83 (m, 4H), 1.99 (m, 22H), 1.69 (s, 3H), 1.54 (s, 9H), 1.52 (s, 3H), 1.23 (s, 3H).

Standards for other derivatized para-hydroquinones are prepared by an analogous methods as would be appreciated by a person of skill in the art.

### Example 2—Alpha-Tocotrienol Hydroquinone Bis-Acetate Ester, Sample Preparation

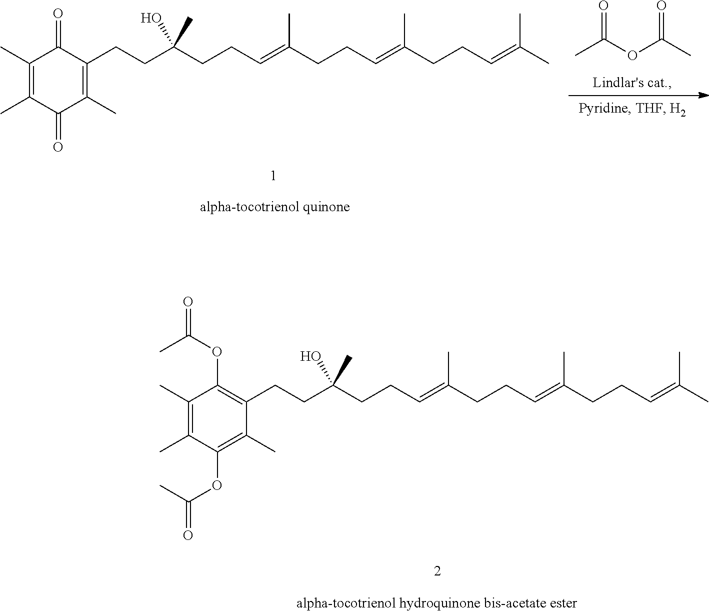

To a 20 mL scintillation vial equipped with a stir bar was added 1 (200 mg, 0.45 mmol), acetic anhydride (129 uL, 1.36 mmol), Lindlar's catalyst (5% wt, 10 mg), pyridine, (365 uL, 4.55 mmol), and THF (3.0 mL, 0.15 M). After the addition was complete, H₂ gas was bubbled through the solution for 1 min and then sealed the vessel was sealed and stirred under H₂ at room temperature overnight.

After stirring overnight, the reaction was stopped and the solution filtered through a syringe filter and concentrated in vacuo at 40° C. and 5 torr. The residue was then re-dissolved with isopropyl acetate, washed with deionized water and saturated aqueous sodium chloride, dried over anhydrous sodium sulfate, filtered, and concentrated in vacuo at 40° C. and 5 torr. About 200 mg of a clear, yellow tint oil was obtained.

Isolation was performed via a silica column, eluting from 0-30% ethyl acetate in heptanes (12 g column monitored at 255 nm). A clear, yellow tint oil was obtained. The oil was then stored at −20° C. overnight to afford 120 mg of a white waxy solid. (50% yield,). Sample yield and purity was obtained by melting point, LC/MS, and NMR. mp=45.4-47.5° C.; LC/MS analysis Rₜ=5.470, (+) m/z=526.75; ¹H-NMR (400 MHz, CD₃OD, 25° C.): δ=5.90 (m, 3H), 2.50 (d, 2H), 2.34 (s, 6H), 1.95 (m, 18H), 1.55 (m, 18H), 1.24 (m, 3H). ¹³C-NMR (400 MHz, CDCl₃, 25° C.): δ=146.09, 145.57, 135.83, 135.22, 131.38, 127.67, 127.60, 126.83, 124.50, 124.17, 72.68, 41.76, 41.10, 39.85, 26.88, 26.50, 25.82, 20.70, 16.16, 13.36, 13.32, 12.59.

### Example 3—Succinate Capping of Alpha-Tocotrienol Hydroquinone in Yeast

In three 125 mL Erlenmeyer flasks, YPD media (25 mL) was inoculated with S. pombe culture (84 μL). Inoculated cultures were shaken in an Amerex Instruments Inc, Gyromax 787 R incubator shaker at 30° C., 220 rpm.

After 16 hrs, cultures were treated with alpha-tocotrienol quinone (1 μM) 5 hours before harvesting. Samples were harvested by transferring to a 50 mL conical tube and spun down in a Eppendorf 5810R centrifuge at 3200 rcf for 3 minutes, 25° C. A sample (2004) was pipetted into a 96-well plate for optical density at 600 nM (OD) reading on a Bio Tek plate reader. The remaining supernatant was poured off and the pellet washed with 0.1% octyl glucoside (OG) (1 mL) in water. Pellets were re-suspended and transferred to a 2 mL correspondingly labeled Eppendorf tube and centrifuged again for 3 min, 22° C. at 3000 rcf in an Eppendorf 5418R centrifuge. The supernatant was removed with vacuum aspiration and the pellet underwent a second wash with 0.1% OG (1 mL) in water. Yeast samples were re-suspended and centrifuged a final time at 3000 g for 3 min at 22° C.; supernatant was removed with vacuum aspiration and the wet yeast pellet was immediately exposed to acyl capping conditions.

Yeast samples were extracted with succinic anhydride (100 mg) in 1 mL 95% acetonitrile 5% triethylamine with internal standard (IS), diclofenac. Samples were vortexed on a VWR analog vortex mixer at a speed of 10 for 30 s and centrifuged in an Eppendorf 5415D at 13.2 rcf, room temperature, for 2.5 min. Supernatant (50 uL) was pulled off and transferred to a 96-well plate and placed in a chilled CTC PAL autosampler at 4° C. for LCMS/MS analysis.

Concentrations/area responses were analyzed using an Eskigent MicroLC 200 Plus with a CTC PAL, and an AB Sciex 6500 QTRAP mass spectrometer. The MS/MS instrument was operated in positive ESI mode, with a source temperature of 250° C. Analytes were separated on a HALO 90A C₁₈ column (0.5×50 mm, 2.7 μm) using a gradient mobile phase method. The composition of mobile phase A was 9:1 0.1% formic acid in water: 0.1% formic acid in methanol. The composition of mobile phase B was 3:1 Acetonitrile: 0.1% formic acid in Isopropyl Alcohol. The MRM transitions were 423.251→165.1 for alpha-tocotrienol quinone, 425.230→165.1 for alpha-tocotrienol hydroquinone, 625.154→265 for alpha-tocotrienol hydroquinone bis-succinate ester, and 295.942→214 for IS-Diclofenac. Data were analyzed using Sciex Analyst Chromatography software, version 1.6.2. The standard curve equation (y=mx+b) is generated from the calibration standards with weighted HILL regression 1/y².

Data from Analyst was normalized for OD and concentrations of components calculated from calibration curves using a linear 1/x² method. Unesterified hydroquinone, or p-HQ, was consistently found to be less than 1% of bis-succinate in all samples. In this study, p-dHQ/(p-dHQ+p-Q) ratio, based on [bis-succinate]/([bis-succinate]+[quinone]) was measured as 0.557+/−0.0012.

### Example 4—Succinate Capping of Alpha-Tocotrienol Hydroquinone in Yeast

In 125 mL Erlenmeyer flasks, 25 mL of YPD media was inoculated with 84 μL of S. Pombe culture in triplicate at the following time points prior to sample harvest: 13 hours, 16 hours, 20 hours, 24 hours, 2 days, 4 days, and 7 days. Inoculated cultures were shaken in an Amerex Instruments Inc, Gyromax 787 R incubator shaker at 30° C., 220 rpm.

All time points were treated with 1 μM of alpha-tocotrienol quinone 5 hours before harvesting. After the 5 hour treatment, yeast growth curve samples were harvested by transferring to a 50 mL conical tube and spun down in a Eppendorf 5810R centrifuge at 3200 rcf for 3 minutes, 25° C. 200 μL of sample was pipetted into a 96-well plate for OD reading on a Bio Tek plate reader. The remaining supernatant was poured off and the pellet washed with 1 mL of 0.1% octyl glucoside (OG) in water. Pellets were re-suspended and transferred to a 2 mL corresponding labeled Eppendorf tube and centrifuged again for 3 minutes, 22° C. at 3000 rcf in an Eppendorf 5418R centrifuge. The supernatant was removed with vacuum aspiration and the pellet underwent 2 additional washes with 1 mL of 0.1% OG in water. Samples were centrifuged a final time at 3000 g for 3 minutes at 22° C.; supernatant removed with vacuum aspiration and the wet yeast pellet was immediately extracted.

Yeast samples along with calibrators were extracted by protein precipitation with 100 mg of succinic anhydride in 1 mL 95% acetonitrile 5% triethylamine with internal standard, diclofenac. Following precipitation, samples were vortexed on a VWR analog vortex mixer at a speed of 10 for 30 seconds and centrifuged in an Eppendorf 5415D at 13.2 rcf, room temperature, for 2.5 min. 50 μL of supernatant was pulled off and transferred to a 96-well plate and placed in a chilled CTC PAL autosampler at 4° C. for LCMS/MS analysis.

Concentrations/area responses were analyzed on an Eskigent MicroLC 200 Plus with a CTC PAL, and an AB Sciex 6500 QTRAP mass spectrometer. The MS/MS instrument was operated in positive ESI mode, with a source temperature of 250° C. Analytes were separated on a HALO 90A C18 column (0.5×50 mm, 2.7 μm) using a gradient mobile phase method. The composition of mobile phase A was 9:1 (0.1% formic acid in water: 0.1% formic acid in methanol. The composition of mobile phase B was 3:1 (Acetonitrile: 0.1% formic acid in Isopropyl Alcohol). The MRM transitions were 423.251→165.1 for alpha-tocotrienol quinone, 425.230→165.1 for alpha tocotrienol hydroquinone, 625.154→265 for alpha tocotrienol hydroquinone bis-succinate ester, and 295.942→214 for IS-Diclofenac. Data were analyzed using Sciex Analyst Chromatography software, version 1.6.2. The standard curve equation (y=mx+b) is generated from the calibration standards with weighted HILL regression 1/y².

Data from analysis were normalized for OD and the ratio of [bis-succinate]/([bis-succinate]+[para-quinone]) and [para-quinone]/[bis-succinate] were plotted. Unesterified hydroquinone was typically found to below 1% of bis-succinate ester.

### Example 5—Clinical Protocol

A clinical trial for determining one or more HQ/TQ or HQ/Q Nernst potentials entails dosing a human cohort with an appropriate pharmaceutical formulation of one or more of the following compounds: alpha-tocotrienol quinone, alpha-tocopherol quinone, coenzyme Q₁₀, vitamin K1, vitamin K2, or another pharmaceutically acceptable quinone; or a hydroquinone thereof. Individual doses may range from 10 to 400 mg alpha-tocotrienol quinone. Individual doses may range from 10 to 400 mg for alpha-tocopherol quinone.

Subjects are monitored closely and at a series of time points, preferably close to the known Cₘₐₓ, and whole blood, plasma and/or white blood cells are drawn. An optional first aliquot of plasma is exposed to atmospheric conditions and any hydroquinone is allowed to auto-oxidized to quinone (“auto-oxidized sample”). A second aliquot of plasma is immediately placed into a sample tube containing acylating solution (“derivatized sample”). The acylating solution contains 100 mg/mL succinic anhydride in 95% acetonitrile: 5% triethylamine. The derivatized sample is vortexed briefly and allowed to incubate for 15-30 minutes at room temperature. Following incubation, precipitated protein is cleared by centrifugation and the derivatized sample is frozen for analysis.

Upon thawing, the derivatized sample and the optional auto-oxidized sample are analyzed for bis-succinate and quinone content using an appropriate LC/MS/MS method.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

1. A compound selected from the group consisting of: 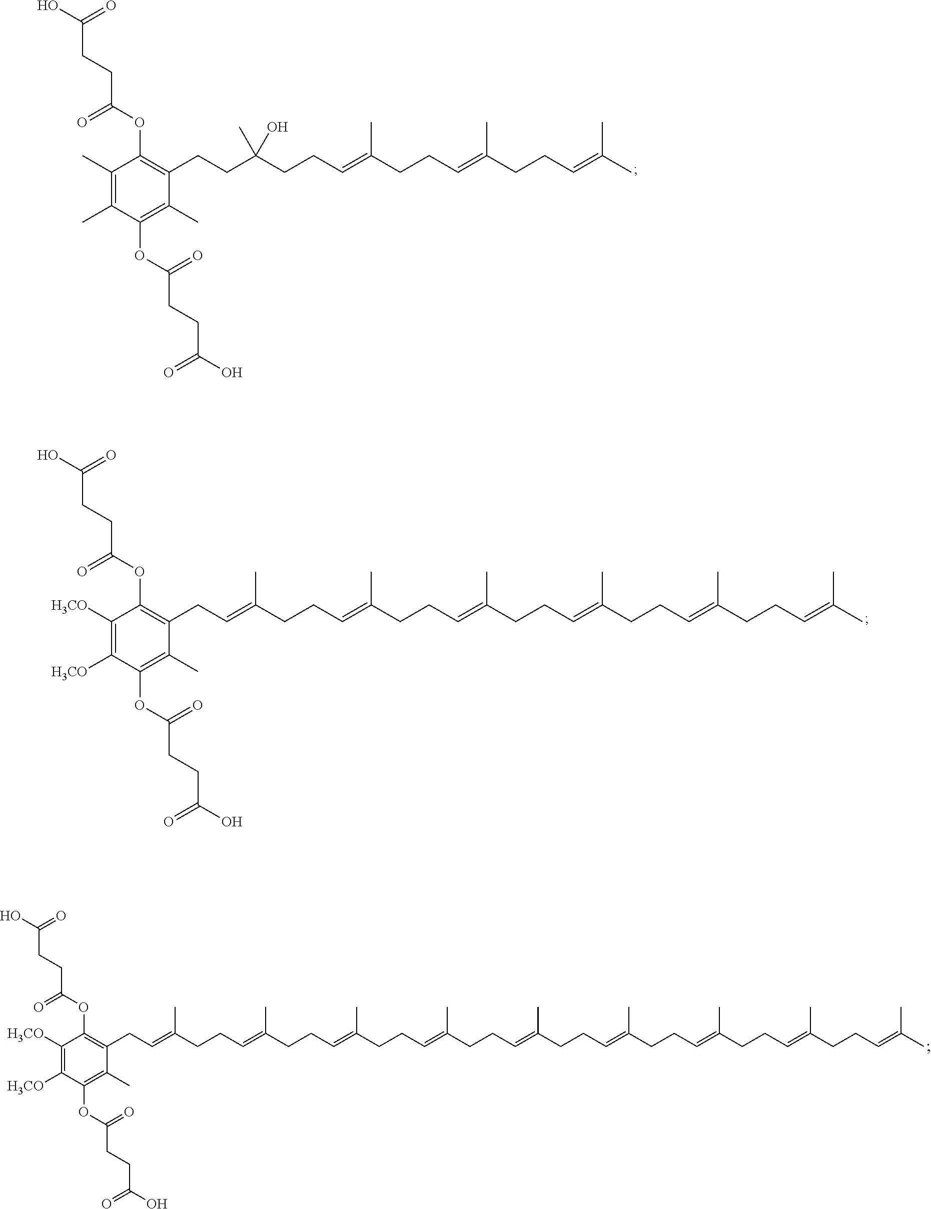 [SMILES: CC(C)=CCC/C(C)=C/CC/C(C)=C/CCC(C)(O)CCc1c(C)c(OC(=O)CCC(=O)O)c(C)c(C)c1OC(=O)CCC(=O)O ; COc1c(OC(=O)CCC(=O)O)c(C)c(C/C=C(\C)CC/C=C(\C)CC/C=C(\C)CC/C=C(\C)CC/C=C(\C)CCC=C(C)C)c(OC(=O)CCC(=O)O)c1OC ; COc1c(OC(=O)CCC(=O)O)c(C)c(C/C=C(\C)CC/C=C(\C)CC/C=C(\C)CC/C=C(\C)CC/C=C(\C)CC/C=C(\C)CC/C=C(\C)CC/C=C(\C)CCC=C(C)C)c(OC(=O)CCC(=O)O)c1OC] 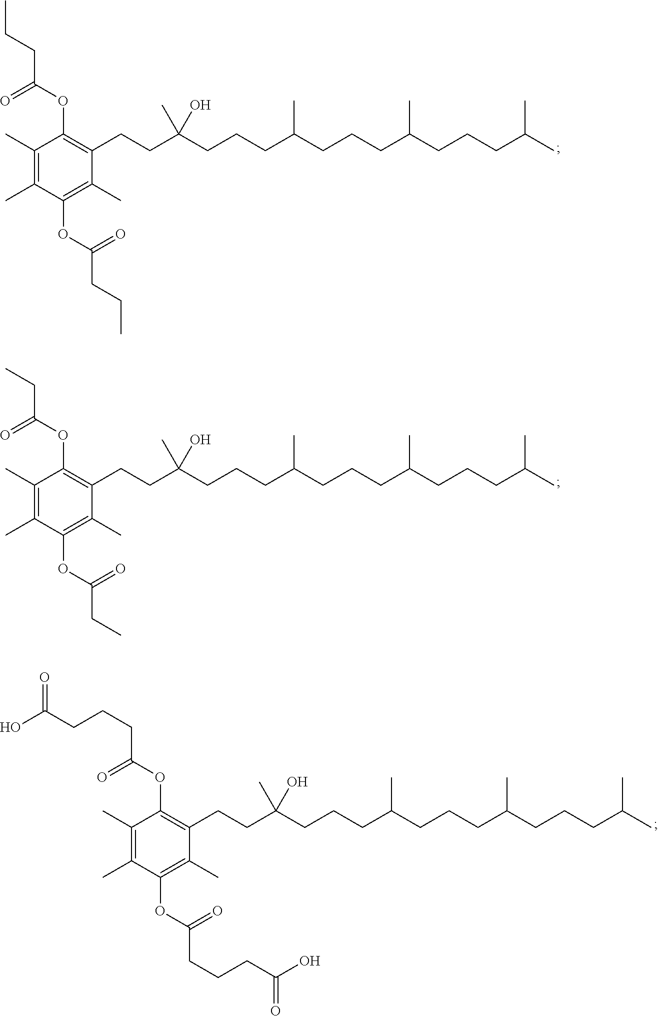 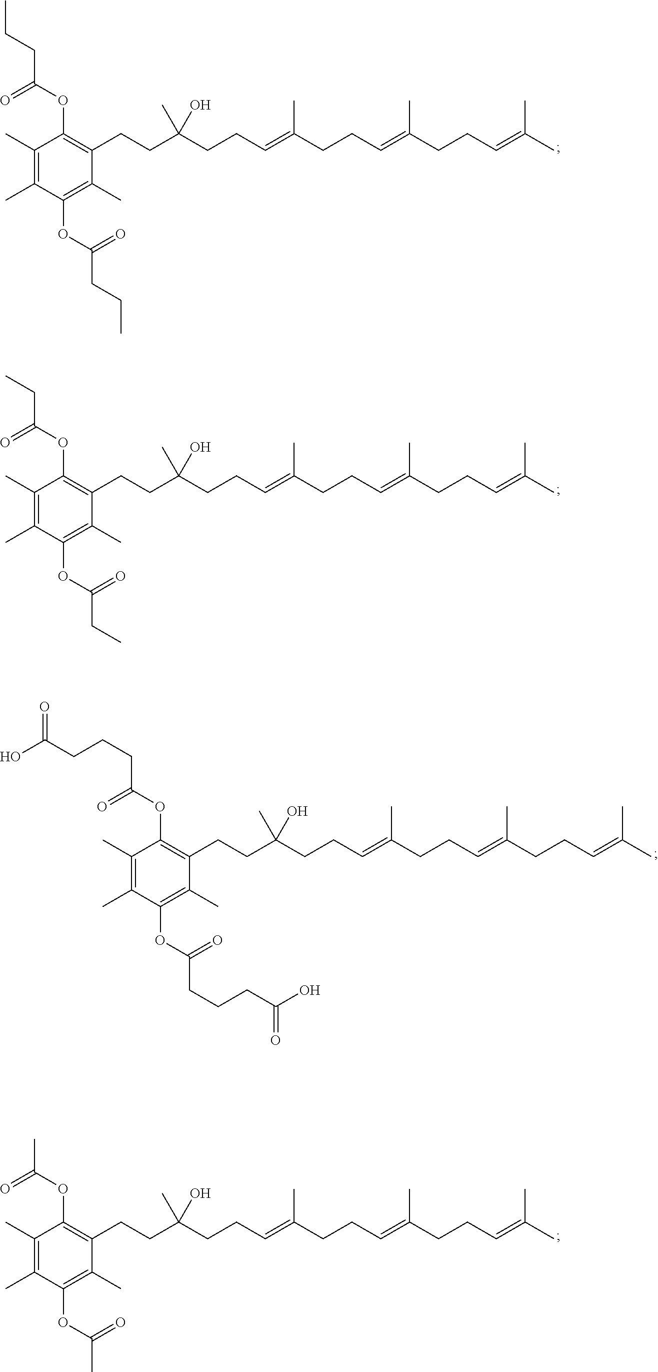 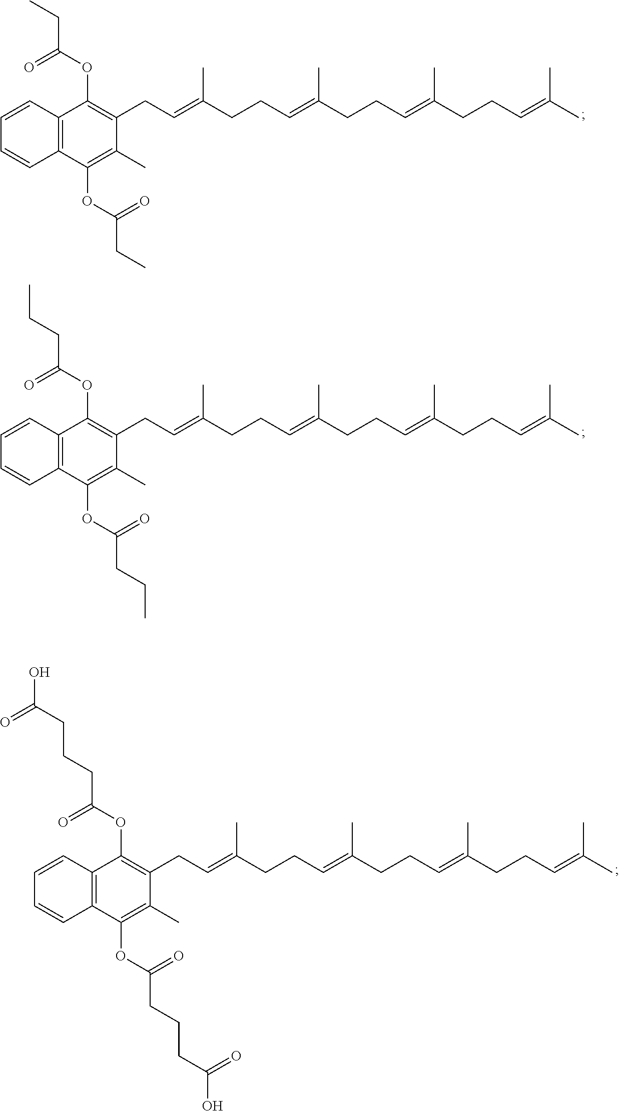 [SMILES: CCC(=O)Oc1c(C)c(C/C=C(\C)CC/C=C(\C)CC/C=C(\C)CCC=C(C)C)c(OC(=O)CC)c2ccccc12 ; CCCC(=O)Oc1c(C)c(C/C=C(\C)CC/C=C(\C)CC/C=C(\C)CCC=C(C)C)c(OC(=O)CCC)c2ccccc12 ; CC(C)=CCC/C(C)=C/CC/C(C)=C/CC/C(C)=C/Cc1c(C)c(OC(=O)CCCC(=O)O)c2ccccc2c1OC(=O)CCCC(=O)O] 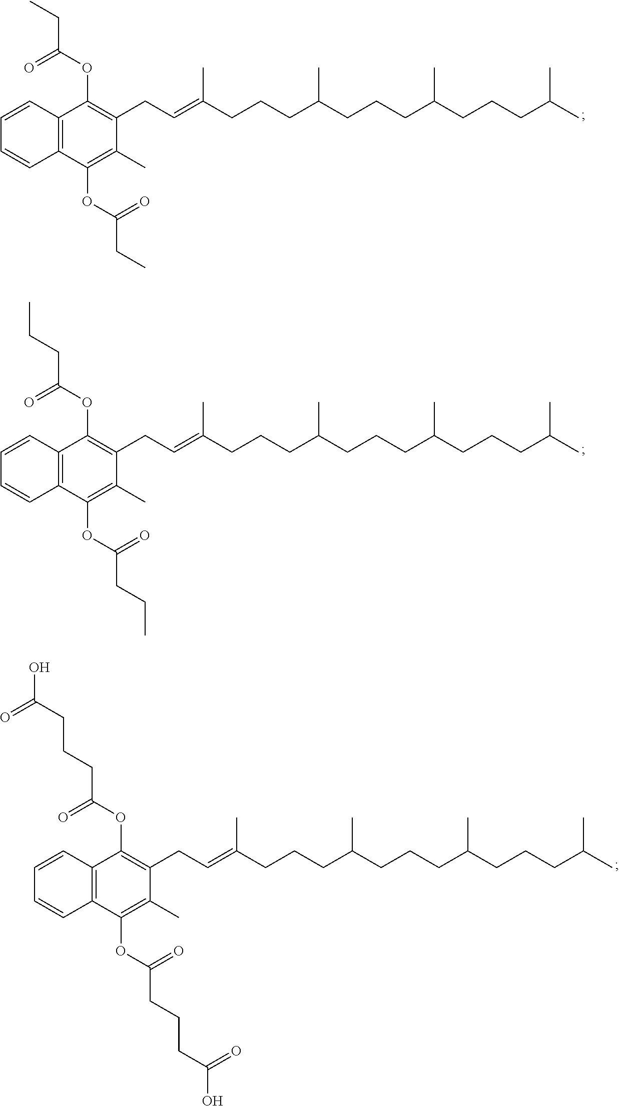 [SMILES: CCC(=O)Oc1c(C)c(C/C=C(\C)CCCC(C)CCCC(C)CCCC(C)C)c(OC(=O)CC)c2ccccc12 ; CCCC(=O)Oc1c(C)c(C/C=C(\C)CCCC(C)CCCC(C)CCCC(C)C)c(OC(=O)CCC)c2ccccc12 ; C/C(=C\Cc1c(C)c(OC(=O)CCCC(=O)O)c2ccccc2c1OC(=O)CCCC(=O)O)CCCC(C)CCCC(C)CCCC(C)C] 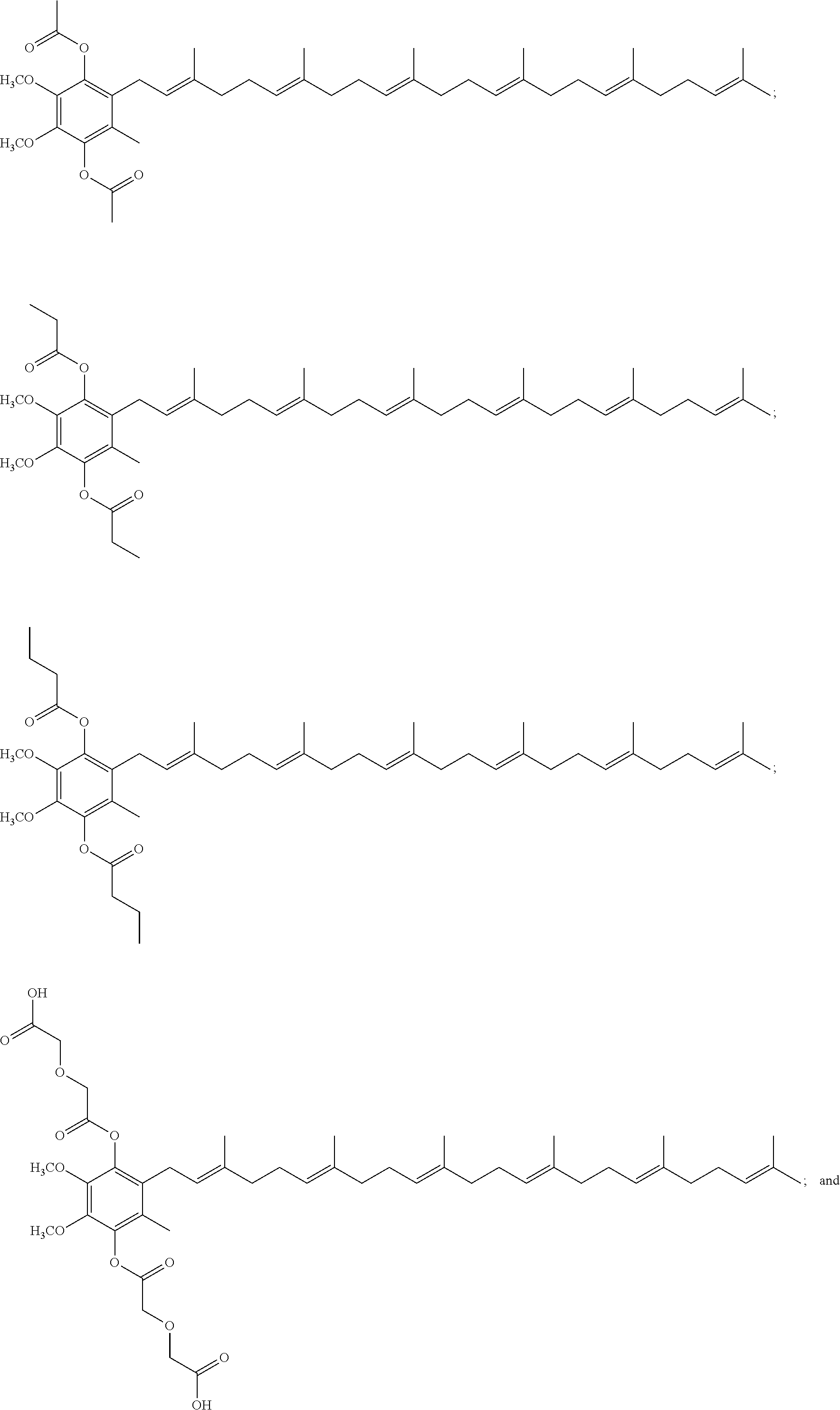 [SMILES: COc1c(OC(C)=O)c(C)c(C/C=C(\C)CC/C=C(\C)CC/C=C(\C)CC/C=C(\C)CC/C=C(\C)CCC=C(C)C)c(OC(C)=O)c1OC ; CCC(=O)Oc1c(C)c(C/C=C(\C)CC/C=C(\C)CC/C=C(\C)CC/C=C(\C)CC/C=C(\C)CCC=C(C)C)c(OC(=O)CC)c(OC)c1OC ; CCCC(=O)Oc1c(C)c(C/C=C(\C)CC/C=C(\C)CC/C=C(\C)CC/C=C(\C)CC/C=C(\C)CCC=C(C)C)c(OC(=O)CCC)c(OC)c1OC ; COc1c(OC(=O)COCC(=O)O)c(C)c(C/C=C(\C)CC/C=C(\C)CC/C=C(\C)CC/C=C(\C)CC/C=C(\C)CCC=C(C)C)c(OC(=O)COCC(=O)O)c1OC] 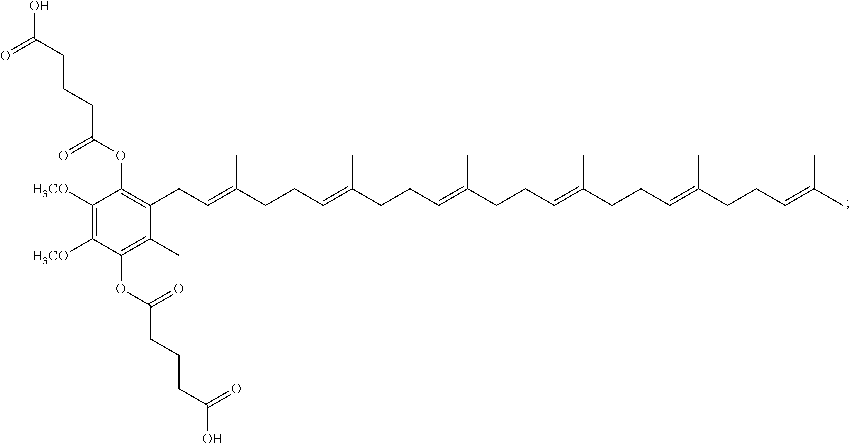 [SMILES: COc1c(OC(=O)CCCC(=O)O)c(C)c(C/C=C(\C)CC/C=C(\C)CC/C=C(\C)CC/C=C(\C)CC/C=C(\C)CCC=C(C)C)c(OC(=O)CCCC(=O)O)c1OC]
or a stereoisomer, a mixture of stereoisomers, and/or a salt thereof.

2. The compound of claim 1 which is 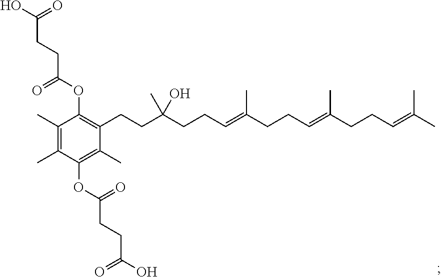 [SMILES: CC(C)=CCC/C(C)=C/CC/C(C)=C/CCC(C)(O)CCc1c(C)c(OC(=O)CCC(=O)O)c(C)c(C)c1OC(=O)CCC(=O)O]
or a stereoisomer, a mixture of stereoisomers, and/or a salt thereof.

3. The compound of claim 1 or 2 which is 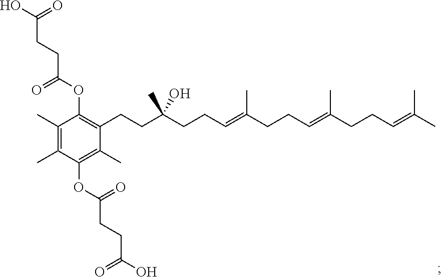 [SMILES: CC(C)=CCC/C(C)=C/CC/C(C)=C/CC[C@@](C)(O)CCc1c(C)c(OC(=O)CCC(=O)O)c(C)c(C)c1OC(=O)CCC(=O)O]
or a salt thereof.

4. The compound of claim 1 which is 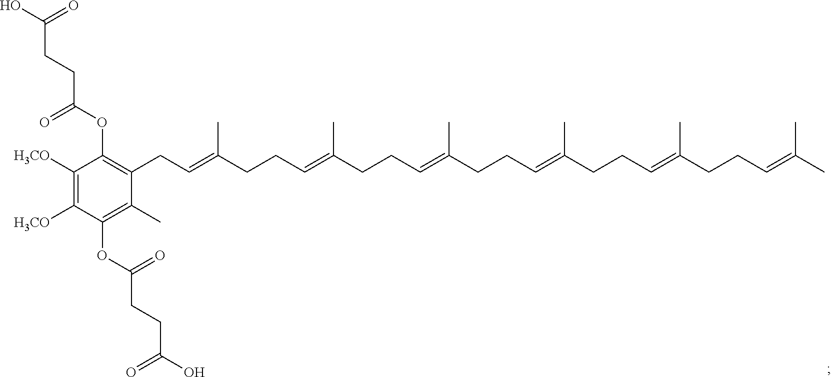 [SMILES: COc1c(OC(=O)CCC(=O)O)c(C)c(C/C=C(\C)CC/C=C(\C)CC/C=C(\C)CC/C=C(\C)CC/C=C(\C)CCC=C(C)C)c(OC(=O)CCC(=O)O)c1OC]
or a salt thereof.

5. The compound of claim 1 which is 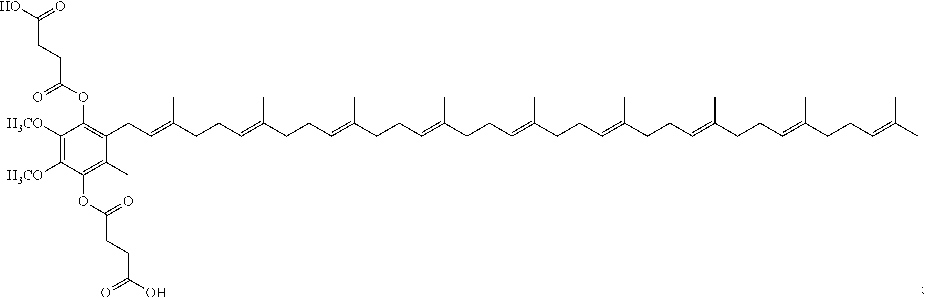 [SMILES: COc1c(OC(=O)CCC(=O)O)c(C)c(C/C=C(\C)CC/C=C(\C)CC/C=C(\C)CC/C=C(\C)CC/C=C(\C)CC/C=C(\C)CC/C=C(\C)CC/C=C(\C)CCC=C(C)C)c(OC(=O)CCC(=O)O)c1OC]
or a salt thereof.

6. (canceled)

7. The compound of claim 1 which is 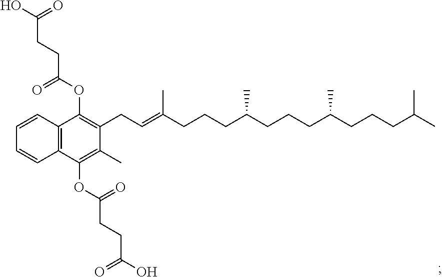 [SMILES: C/C(=C\Cc1c(C)c(OC(=O)CCC(=O)O)c2ccccc2c1OC(=O)CCC(=O)O)CCC[C@H](C)CCC[C@H](C)CCCC(C)C]
or a salt thereof.

8. A method of determining the level of one or more para-hydroquinones in a first sample, the method comprising:
acylating the one or more para-hydroquinones in the first sample to one or more para-hydroquinone derivatives by treatment with an alkyl carboxylic acid, an activated alkyl carboxylic acid, an alkyl anhydride, or a cyclic anhydride; and
determining the level of the one or more para-hydroquinone derivatives in the first sample.

9. The method of claim 8 wherein the one or more para-hydroquinones in the first sample are acylated with an alkyl anhydride or a cyclic anhydride.

10. The method of claim 8 or 9 wherein the one or more para-hydroquinone derivatives are selected from the group consisting of a bis-acetate derivative, a bis-propionate derivative, a bis-butyrate derivative, a bis-succinate derivative, a bis-glutarate derivative, and a bis-diglycolate derivative.

11. The method of any one of claims 8-10 wherein the one or more para-hydroquinone derivatives are bis-acetate derivatives.

12. The method of any one of claims 8-10 wherein the one or more para-hydroquinone derivatives are bis-propionate derivatives.

13. The method of any one of claims 8-10 wherein the one or more para-hydroquinone derivatives are bis-butyrate derivatives.

14. The method of any one of claims 8-10 wherein the one or more para-hydroquinone derivatives are bis-succinate derivatives.

15. The method of any one of claims 8-10 wherein the one or more para-hydroquinone derivatives are bis-glutarate derivatives.

16. The method of any one of claims 8-10 wherein the one or more para-hydroquinone derivatives are bis-diglycolate derivatives.

17. The method of any one of claims 8-16, further comprising determining the level of one or more corresponding para-quinones in an optional second sample, taken at the same time as the first sample, wherein the one or more para-hydroquinones in the second sample was allowed to auto-oxidize to the one or more corresponding para-quinones.

18. A method of determining the ratio or reverse ratio of one or more para-hydroquinones to one or more corresponding para-quinones in a first sample, comprising:
(i) determining the level of one or more para-hydroquinone derivatives of the one or more para-hydroquinones in the first sample according to any one of claims 8-16; and
(ii) determining one or more of the following (a)-(d):
(a) the sum of the level of the one or more para-hydroquinone derivatives and the level of the one or more corresponding para-quinones in the first sample;
(b) the level of one or more corresponding para-quinones in the first sample;
(c) the level of total quinones in the first sample; and
(d) the level of one or more corresponding para-quinones in an optional second sample, taken at the same time as the first sample, wherein the one or more para-hydroquinones in the second sample were allowed to auto-oxidize to the one or more corresponding para-quinones; and
(iii) determining a ratio or reverse ratio of one or more of the following (a)-(d):
(a) the level of the one or more para-hydroquinone derivatives in the first sample to the sum of the level of the one or more para-hydroquinone derivatives and the level of the one or more corresponding para-quinones in the first sample;
(b) the level of the one or more para-hydroquinone derivatives in the first sample to the level of the one or more corresponding para-quinones in the first sample;
(c) the level of the one or more para-hydroquinone derivatives in the first sample to the level of the total quinones in the first sample; and
(d) the level of the one or more para-hydroquinone derivatives in the first sample to the level of the one or more corresponding para-quinones in the second sample.

19. The method of claim 18 wherein the ratio or reverse ratio of the level of the one or more para-hydroquinone derivatives in the first sample to the sum of the level of the one or more para-hydroquinone derivatives and the level of the one or more corresponding para-quinones in the first sample is determined.

20. The method of claim 18 wherein the ratio or reverse ratio of the level of the one or more para-hydroquinone derivatives in the first sample to the level of the one or more corresponding para-quinones in the first sample is determined.

21. The method of claim 18 wherein the ratio or reverse ratio of the level of the one or more para-hydroquinone derivatives in the first sample to the level of the total quinones in the first sample is determined.

22. The method of claim 18 wherein the ratio or reverse ratio of the level of the one or more para-hydroquinone derivatives in the first sample to the level of the one or more corresponding para-quinones in the second sample is determined.

23. The method of any one of claims 8-22 where the levels are determined by mass spectroscopy.

24. The method of any one of claims 8-22 where the levels are determined by liquid chromatography-mass spectroscopy (LC-MS).

25. The method of any one of claims 8-24 wherein the first sample and the optional second sample are biological samples.

26. The method of claim 25 wherein the biological samples are selected from the group consisting of a cell sample, bacterial culture, yeast culture, plasma, serum, whole blood, and a biological tissue sample.

27. The method of any one of claims 8-24 wherein the first sample and optional second sample are food samples.

28. The method of any one of claims 8-27 wherein the level(s) which are determined are mass(es), moles, or concentration(s).

29. The method of any one of claims 8-28 wherein the one or more para-hydroquinone derivatives are selected from the group consisting of 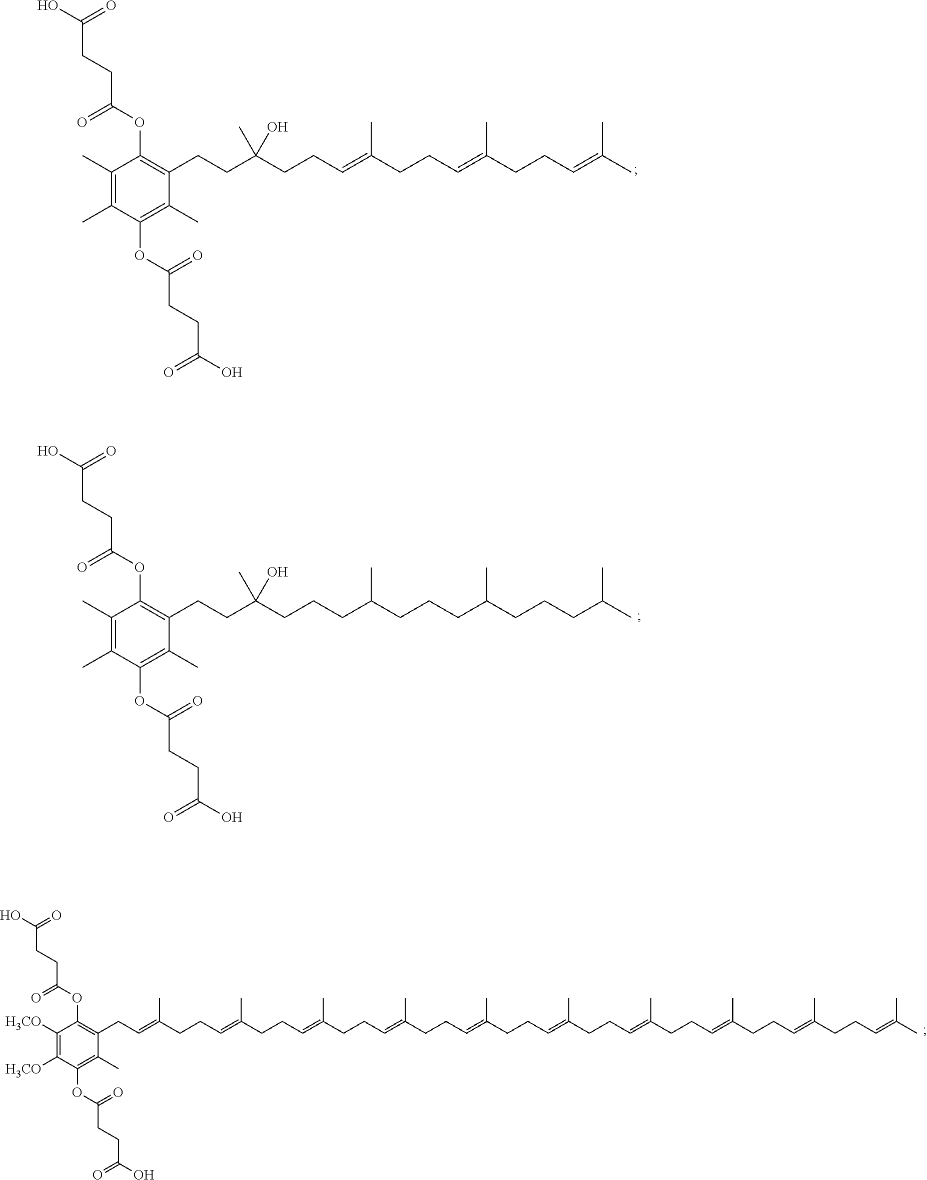 [SMILES: CC(C)=CCC/C(C)=C/CC/C(C)=C/CCC(C)(O)CCc1c(C)c(OC(=O)CCC(=O)O)c(C)c(C)c1OC(=O)CCC(=O)O ; Cc1c(C)c(OC(=O)CCC(=O)O)c(CCC(C)(O)CCCC(C)CCCC(C)CCCC(C)C)c(C)c1OC(=O)CCC(=O)O ; COc1c(OC(=O)CCC(=O)O)c(C)c(C/C=C(\C)CC/C=C(\C)CC/C=C(\C)CC/C=C(\C)CC/C=C(\C)CC/C=C(\C)CC/C=C(\C)CC/C=C(\C)CC/C=C(\C)CCC=C(C)C)c(OC(=O)CCC(=O)O)c1OC] 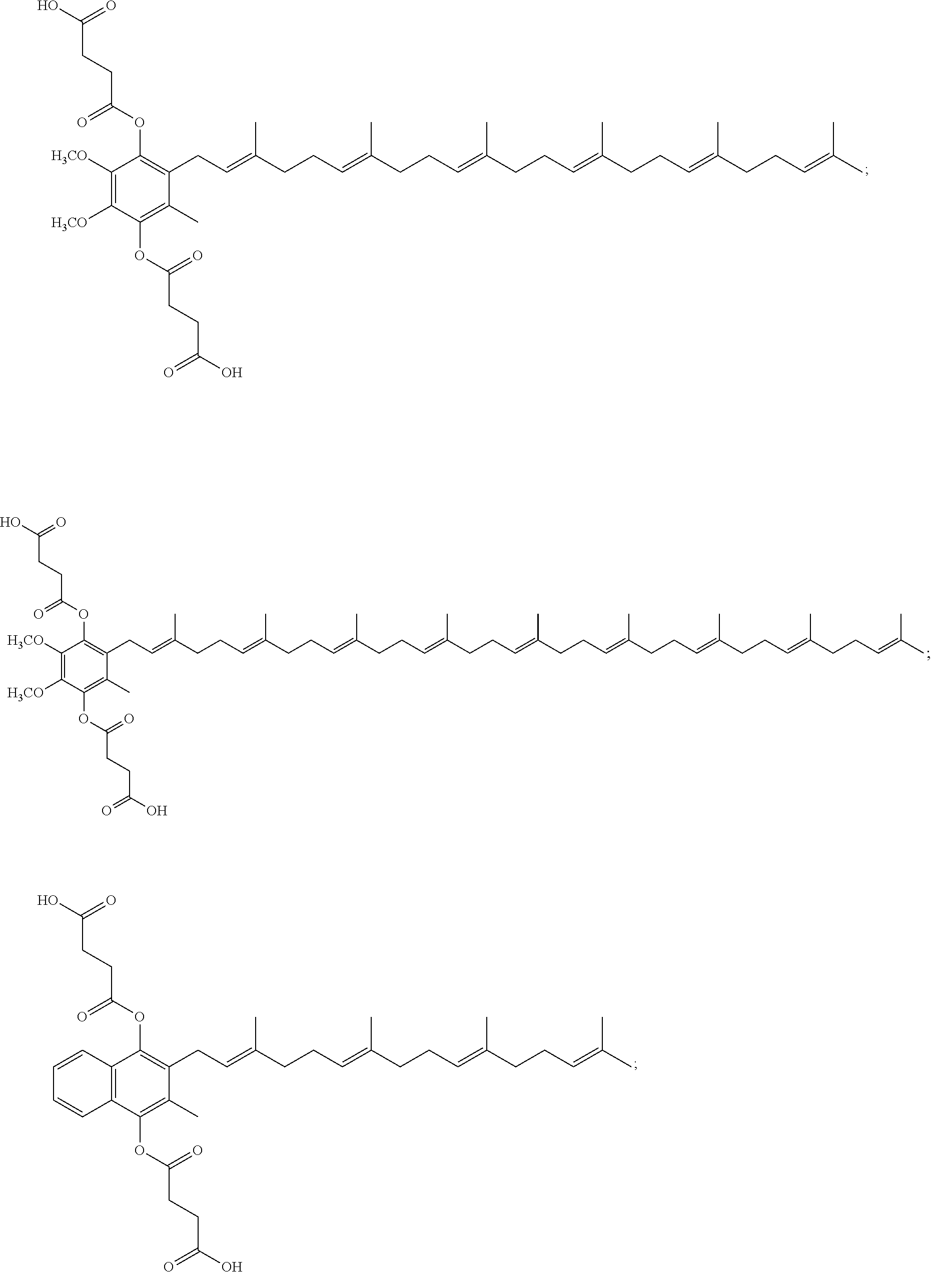 [SMILES: COc1c(OC(=O)CCC(=O)O)c(C)c(C/C=C(\C)CC/C=C(\C)CC/C=C(\C)CC/C=C(\C)CC/C=C(\C)CCC=C(C)C)c(OC(=O)CCC(=O)O)c1OC ; COc1c(OC(=O)CCC(=O)O)c(C)c(C/C=C(\C)CC/C=C(\C)CC/C=C(\C)CC/C=C(\C)CC/C=C(\C)CC/C=C(\C)CC/C=C(\C)CC/C=C(\C)CCC=C(C)C)c(OC(=O)CCC(=O)O)c1OC ; CC(C)=CCC/C(C)=C/CC/C(C)=C/CC/C(C)=C/Cc1c(C)c(OC(=O)CCC(=O)O)c2ccccc2c1OC(=O)CCC(=O)O] 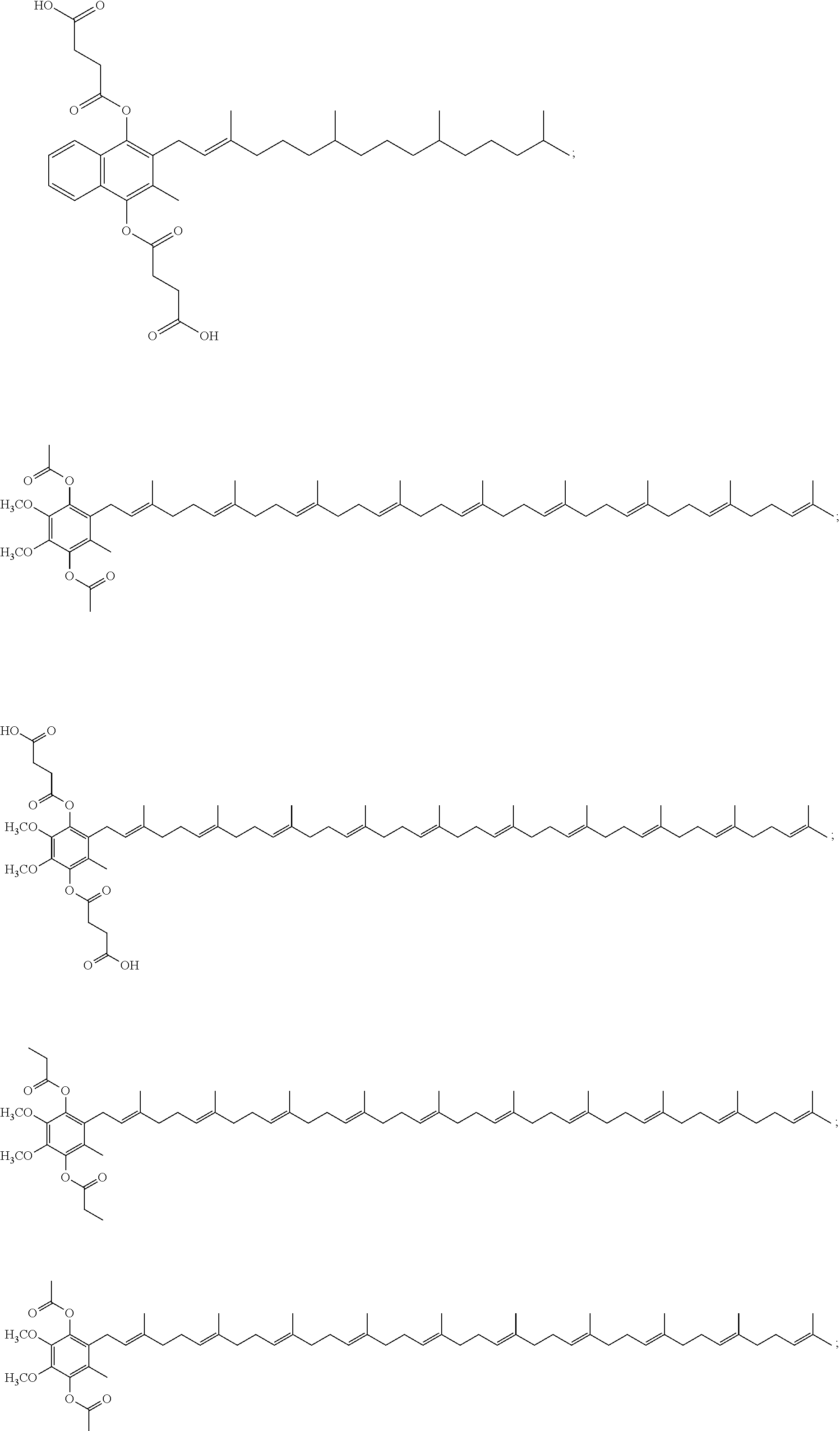 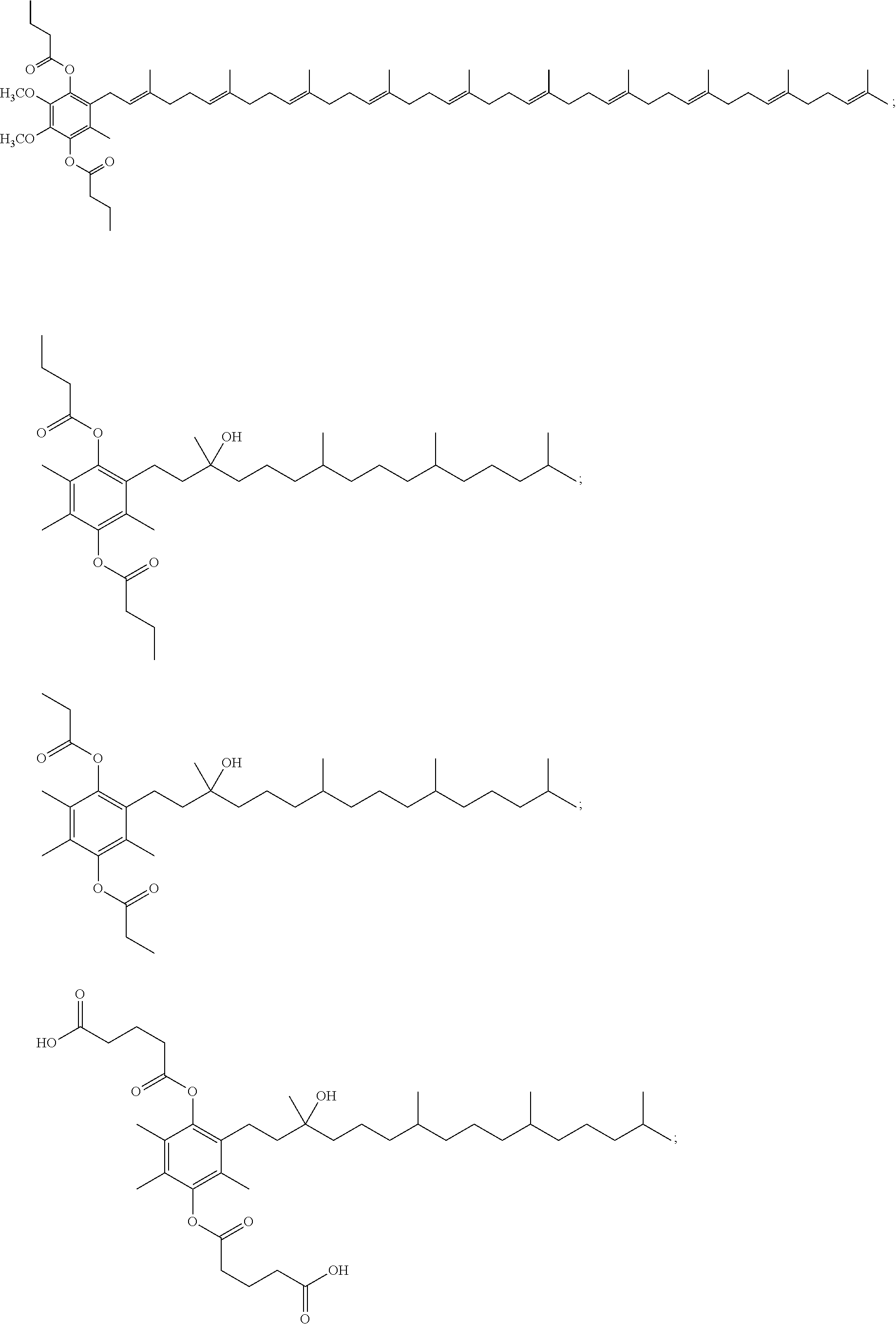 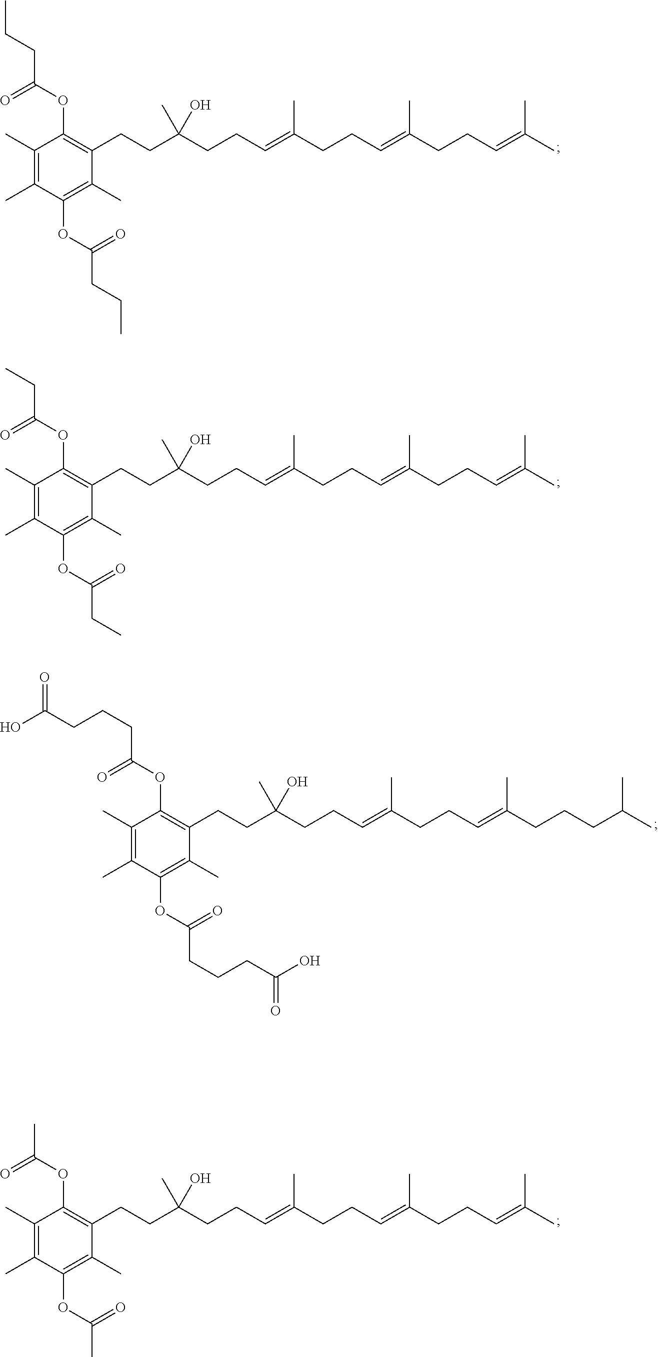 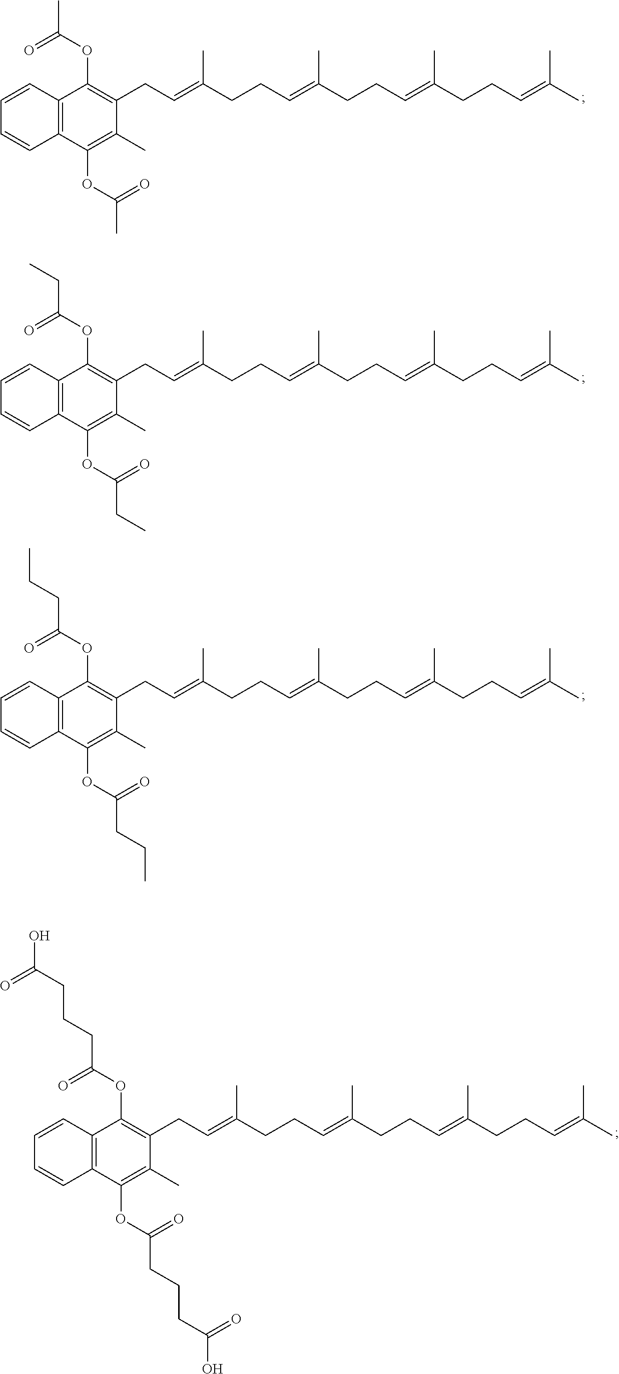 [SMILES: CC(=O)Oc1c(C)c(C/C=C(\C)CC/C=C(\C)CC/C=C(\C)CCC=C(C)C)c(OC(C)=O)c2ccccc12 ; CCC(=O)Oc1c(C)c(C/C=C(\C)CC/C=C(\C)CC/C=C(\C)CCC=C(C)C)c(OC(=O)CC)c2ccccc12 ; CCCC(=O)Oc1c(C)c(C/C=C(\C)CC/C=C(\C)CC/C=C(\C)CCC=C(C)C)c(OC(=O)CCC)c2ccccc12 ; CC(C)=CCC/C(C)=C/CC/C(C)=C/CC/C(C)=C/Cc1c(C)c(OC(=O)CCCC(=O)O)c2ccccc2c1OC(=O)CCCC(=O)O] 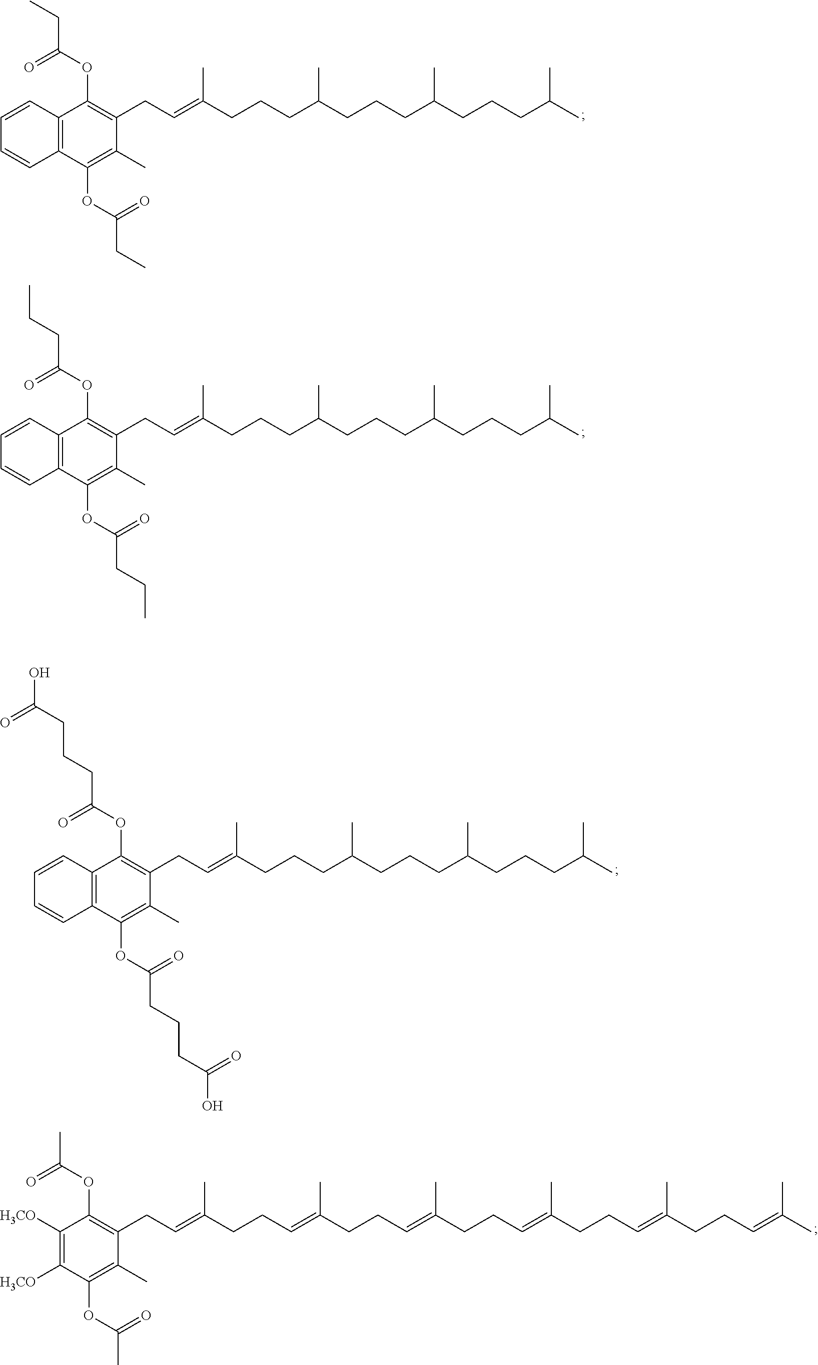 [SMILES: CCC(=O)Oc1c(C)c(C/C=C(\C)CCCC(C)CCCC(C)CCCC(C)C)c(OC(=O)CC)c2ccccc12 ; CCCC(=O)Oc1c(C)c(C/C=C(\C)CCCC(C)CCCC(C)CCCC(C)C)c(OC(=O)CCC)c2ccccc12 ; C/C(=C\Cc1c(C)c(OC(=O)CCCC(=O)O)c2ccccc2c1OC(=O)CCCC(=O)O)CCCC(C)CCCC(C)CCCC(C)C ; COc1c(OC(C)=O)c(C)c(C/C=C(\C)CC/C=C(\C)CC/C=C(\C)CC/C=C(\C)CC/C=C(\C)CCC=C(C)C)c(OC(C)=O)c1OC] 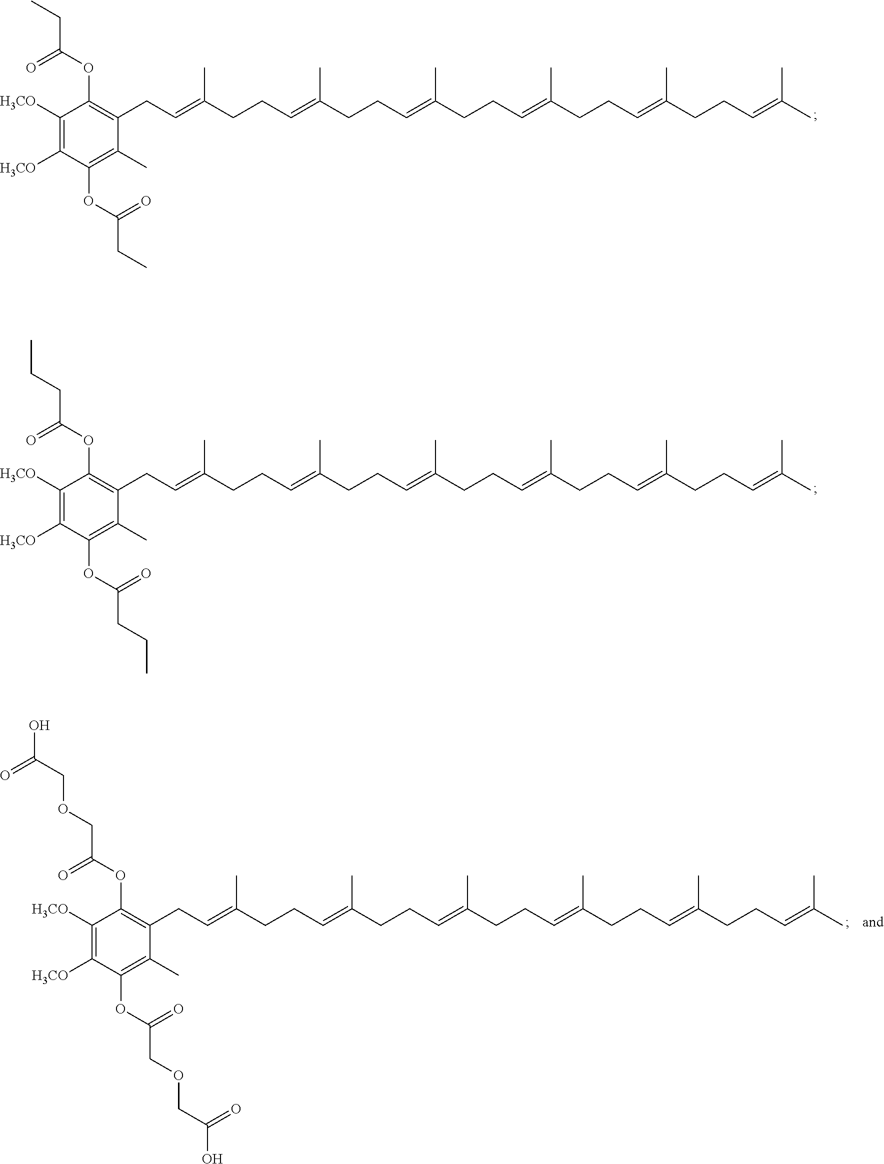 [SMILES: CCC(=O)Oc1c(C)c(C/C=C(\C)CC/C=C(\C)CC/C=C(\C)CC/C=C(\C)CC/C=C(\C)CCC=C(C)C)c(OC(=O)CC)c(OC)c1OC ; CCCC(=O)Oc1c(C)c(C/C=C(\C)CC/C=C(\C)CC/C=C(\C)CC/C=C(\C)CC/C=C(\C)CCC=C(C)C)c(OC(=O)CCC)c(OC)c1OC ; COc1c(OC(=O)COCC(=O)O)c(C)c(C/C=C(\C)CC/C=C(\C)CC/C=C(\C)CC/C=C(\C)CC/C=C(\C)CCC=C(C)C)c(OC(=O)COCC(=O)O)c1OC] 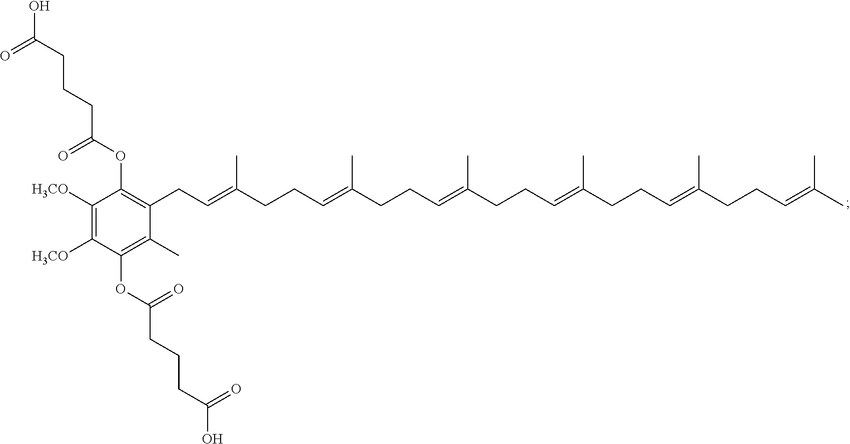 [SMILES: COc1c(OC(=O)CCCC(=O)O)c(C)c(C/C=C(\C)CC/C=C(\C)CC/C=C(\C)CC/C=C(\C)CC/C=C(\C)CCC=C(C)C)c(OC(=O)CCCC(=O)O)c1OC]
or a stereoisomer, a mixture of stereoisomers, and/or a salt thereof.

30. The method of claim 29 wherein the one or more para-hydroquinone derivatives comprise 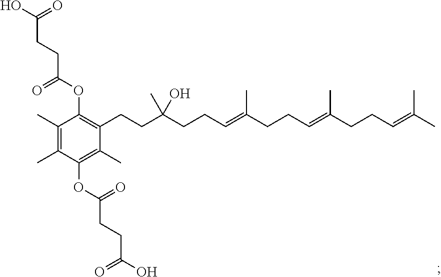 [SMILES: CC(C)=CCC/C(C)=C/CC/C(C)=C/CCC(C)(O)CCc1c(C)c(OC(=O)CCC(=O)O)c(C)c(C)c1OC(=O)CCC(=O)O]
or a stereoisomer, a mixture of stereoisomers, and/or a salt thereof.

31. The method of claim 29 wherein the one or more para-hydroquinone derivatives comprise 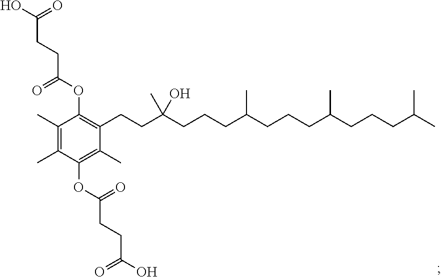 [SMILES: Cc1c(C)c(OC(=O)CCC(=O)O)c(CCC(C)(O)CCCC(C)CCCC(C)CCCC(C)C)c(C)c1OC(=O)CCC(=O)O]
or a stereoisomer, a mixture of stereoisomers, and/or a salt thereof.

32. The method of claim 29 wherein the one or more para-hydroquinone derivatives comprise 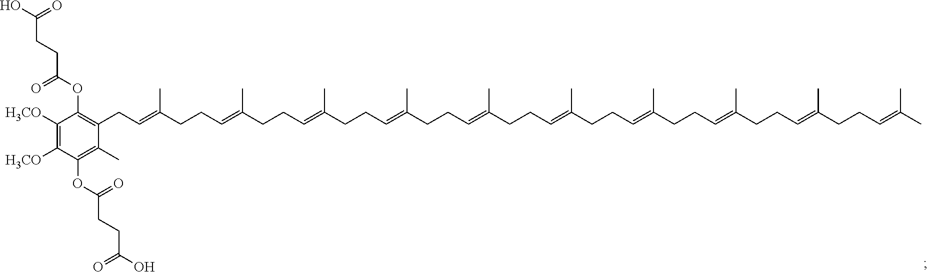 [SMILES: COc1c(OC(=O)CCC(=O)O)c(C)c(C/C=C(\C)CC/C=C(\C)CC/C=C(\C)CC/C=C(\C)CC/C=C(\C)CC/C=C(\C)CC/C=C(\C)CC/C=C(\C)CC/C=C(\C)CCC=C(C)C)c(OC(=O)CCC(=O)O)c1OC]
or a salt thereof.

33. The method of claim 29 wherein the one or more para-hydroquinone derivatives comprise 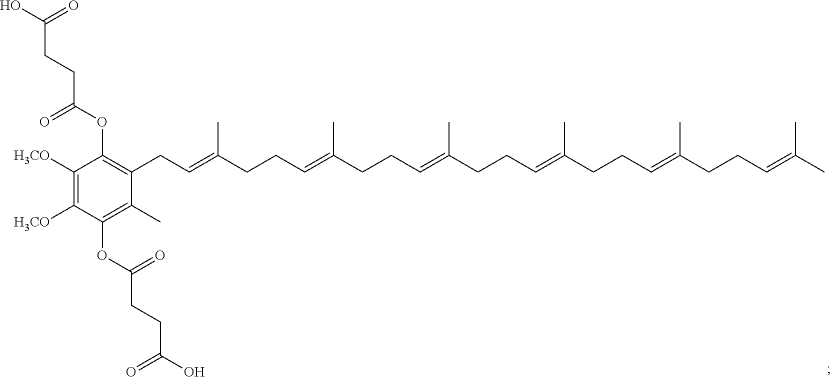 [SMILES: COc1c(OC(=O)CCC(=O)O)c(C)c(C/C=C(\C)CC/C=C(\C)CC/C=C(\C)CC/C=C(\C)CC/C=C(\C)CCC=C(C)C)c(OC(=O)CCC(=O)O)c1OC]
or a salt thereof.

34. The method of claim 29 wherein the one or more para-hydroquinone derivatives comprise 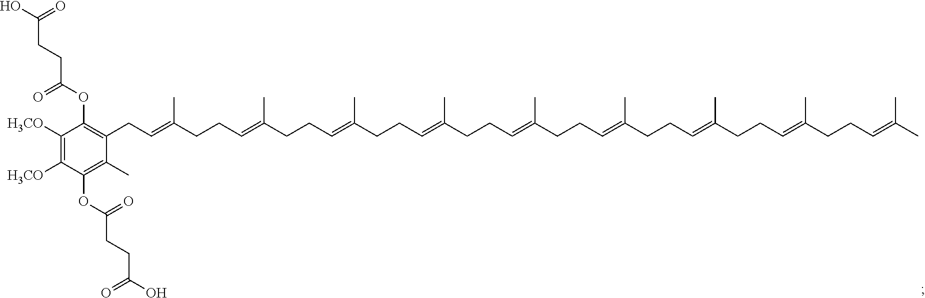 [SMILES: COc1c(OC(=O)CCC(=O)O)c(C)c(C/C=C(\C)CC/C=C(\C)CC/C=C(\C)CC/C=C(\C)CC/C=C(\C)CC/C=C(\C)CC/C=C(\C)CC/C=C(\C)CCC=C(C)C)c(OC(=O)CCC(=O)O)c1OC]
or a salt thereof.

35. The method of claim 29 wherein the one or more para-hydroquinone derivatives comprise 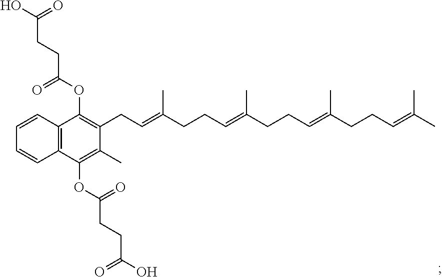 [SMILES: CC(C)=CCC/C(C)=C/CC/C(C)=C/CC/C(C)=C/Cc1c(C)c(OC(=O)CCC(=O)O)c2ccccc2c1OC(=O)CCC(=O)O]
or a salt thereof.

36. The method of claim 29 wherein the one or more para-hydroquinone derivatives comprise 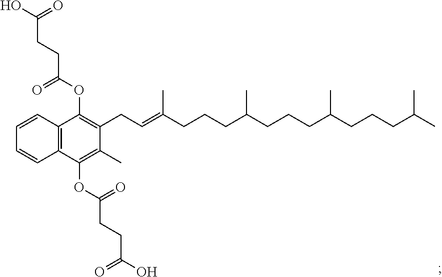 [SMILES: C/C(=C\Cc1c(C)c(OC(=O)CCC(=O)O)c2ccccc2c1OC(=O)CCC(=O)O)CCCC(C)CCCC(C)CCCC(C)C]
or a stereoisomer, a mixture of stereoisomers, and/or a salt thereof.

37. The method of claim 29 or 30 wherein the one or more para-hydroquinone derivatives comprise 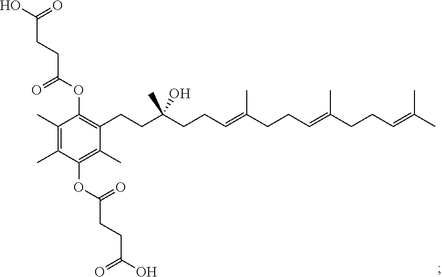 [SMILES: CC(C)=CCC/C(C)=C/CC/C(C)=C/CC[C@@](C)(O)CCc1c(C)c(OC(=O)CCC(=O)O)c(C)c(C)c1OC(=O)CCC(=O)O]
or a salt thereof.

38. The method of claim 29 or 31 wherein the one or more para-hydroquinone derivatives comprise 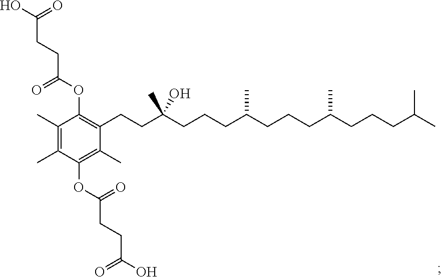 [SMILES: Cc1c(C)c(OC(=O)CCC(=O)O)c(CC[C@](C)(O)CCC[C@H](C)CCC[C@H](C)CCCC(C)C)c(C)c1OC(=O)CCC(=O)O]
or a salt thereof.

39. The method of claim 29 or 36 wherein the one or more para-hydroquinone derivatives comprise 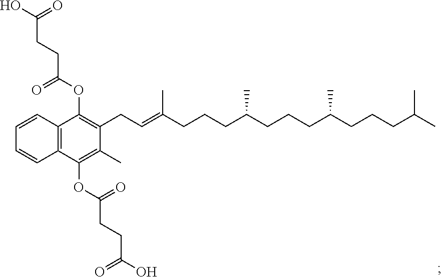 [SMILES: C/C(=C\Cc1c(C)c(OC(=O)CCC(=O)O)c2ccccc2c1OC(=O)CCC(=O)O)CCC[C@H](C)CCC[C@H](C)CCCC(C)C]
or a salt thereof.
